(19) United States
(12) Patent Application Publication
(10) Pub. No.: US 2025/0154143 A1
(43) Pub. Date: May 15, 2025

(54) PRODRUGS FOR USE IN THE TREATMENT OF TISSUE DAMAGE

(71) Applicant: UCL BUSINESS LTD, London (GB)

(72) Inventors: Mark Brian PEPYS, London (GB); Melanie Susanne GLOSSOP, Stevenage (GB); Christopher SWAIN, London (GB); Graham Walter TAYLOR, London (GB); Stephen Paul WOOD, London (GB)

(21) Appl. No.: 18/683,412

(22) PCT Filed: Aug. 18, 2022
(86) PCT No.: PCT/EP2022/073107

§ 371 Date: Feb. 13, 2024

(30) Foreign Application Priority Data
Aug. 18, 2021 (GB) 2111866.6

(51) Int. Cl. C07D 453/02

(52) U.S. Cl. CPC C07D 453/02

(57) ABSTRACT

An agent for use in medicine, wherein the agent comprises a compound of Formula (I): wherein: Ar is an aryl linker group, and R¹ and R² are independently selected from groups of formula —OH; —OR³; —O(CO)R³; —NR³R^{3′}; wherein: R³ and R^{3′} are independently selected from H, C1-C12 alkyl, C1-C12 alkylene, C3-C8 (hetero)cycloalkyl, or C5-C12 (hetero)aryl, or R3 and R^{3′} together form a C3-C8 heterocyclic ring with the nitrogen, each optionally substituted with one, two or three groups selected from halogen, —CF₃, —OR⁶, —NR⁶R⁶, —COR⁶, and —CO₂R⁶, C3-C8 (hetero)cycloalkyl, or C5-C12 (hetero)aryl; R⁴ and R⁵ are independently selected from H or C1-C6 alkyl; and R⁶ and R⁶ are independently C1-C6 alkyl, or together form a C3-C8 heterocyclic ring with the nitrogen, provided that R¹ and R² are not both —OH, including individual pharmaceutically acceptable salts, solvates, or derivatives thereof.

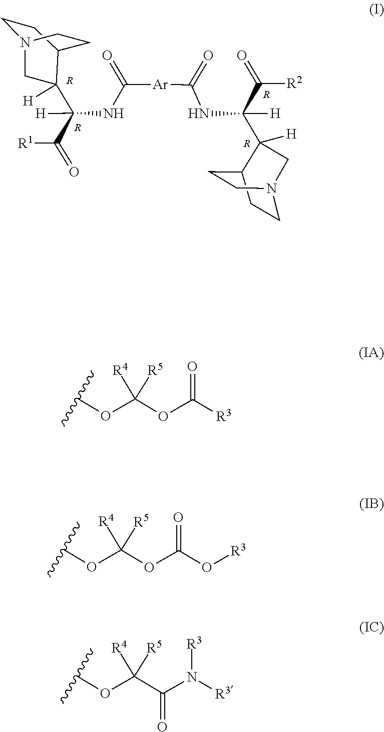

### FIELD OF THE INVENTION

The present invention relates to prodrugs that release agents that are specifically bound by C-reactive protein (CRP) in vivo, thereby inhibiting the binding of CRP to autologous cellular and tissue ligands, and to compositions containing such agents for use in the treatment or prevention of tissue damage, in particular in ischaemic, traumatic, infectious, inflammatory and neoplastic conditions.

### BACKGROUND OF THE INVENTION

C-reactive protein (CRP) is a normal plasma protein of the pentraxin protein family, the other member of which is serum amyloid P component (SAP) (1). CRP is the classical acute phase protein, the circulating concentration of which increases dramatically in response to most forms of tissue injury, infection, inflammation and cancer. In most conditions the CRP value attained correlates closely with the extent and severity of disease. CRP is a calcium dependent ligand binding protein, which binds with highest affinity to phosphocholine residues, though it also binds a variety of other ligands of both autologous and extrinsic origin. Autologous ligands include native and modified plasma lipoproteins, damaged cell membranes, a number of different phospholipids and related compounds, and small nuclear ribonucleoprotein particles. Extrinsic ligands include some glycan, phospholipid and other components of micro-organisms, such as capsular and somatic components of bacteria, fungi and parasites, as well as plant products. CRP bound to macromolecular ligands activates the classical complement pathway via Clq, leading to activation and fixation of C3, the main adhesion molecule of the complement system, production of the major chemotactic factors, C3a and C5a, and engagement of the terminal lytic phase, C5-C9.

In addition to closely reflecting the extent and activity of whatever disease process has triggered increased CRP production, higher circulating concentrations of CRP also significantly predict progression of disease, incidence of complications and poorer clinical outcome. Extensive clinical observations of this association, across a wide spectrum of diseases, are consistent with a pathogenic role of CRP in exacerbating tissue damage and thus disease severity. CRP does not bind to normal healthy cells but binds avidly to ligands exposed on dead and damaged cells and it then activates complement. Whilst CRP-mediated complement activation may contribute to clearance of cellular debris from the tissues and to host defence against some micro-organisms, it is clear that, just as in many antibody-mediated hyper-sensitivity reactions, complement activation can cause severe tissue damage.

The complement dependent pathogenicity of human CRP was first confirmed experimentally by the demonstration that administration of human CRP to rats undergoing coronary artery ligation increased the size of the resulting acute myocardial infarcts (2). Human CRP and activated rat complement were deposited in and around the infarct and the exacerbation of tissue damage was absolutely complement dependent. Similar observations were made in the middle cerebral artery occlusion model of stroke in rats (3). Subsequently several different independent groups have made comparable observations in a range of different animal models.

The design of the first small molecule inhibitor of CRP binding for use in vivo, bis(phosphocholine)hexane (BPC6), enabled conclusive confirmation of the pathogenic role of human CRP in exacerbating tissue damage after ischaemic infarction (2). Administration of this compound to rats undergoing coronary artery ligation and receiving human CRP completely abrogated the increased damage that occurred in human CRP treated animals which did not receive the treatment. Subsequently, bis(phosphocholine)octane (BPC8), was found to be a more potent inhibitor of CRP binding in vitro and it had the same protective effect against human CRP pathogenicity in the rat acute myocardial infarction model, including the ischaemia reperfusion design as well as after terminal coronary artery ligation (Pepys, unpublished observations). Human CRP was thus validated as a therapeutic target and efficacy of intervention via a small molecule inhibitor of CRP binding was demonstrated.

These observations opened the way to a novel avenue for reducing disease severity in the very wide variety of tissue damaging conditions in which there are increased circulating concentrations of CRP. Inhibition of CRP binding in vivo will obviously not prevent or cure diverse diseases with very different aetiologies. However, reducing the extent, severity and duration of tissue damage and thus prolonging survival in patients with heart attacks, strokes, rheumatoid arthritis and other chronic inflammatory disease of unknown cause, burns, bacterial and viral infections or cancer cachexia, and many other conditions, remains an urgent major unmet medical need.

WO03/097104 A1 describes an agent that is bound by CRP and inhibits CRP binding to other ligands. The agent comprises a plurality of ligands covalently co-linked so as to form a complex with a plurality of C-reactive protein (CRP) molecules, wherein (i) at least two of the ligands are the same or different and are capable of being bound by ligand binding sites present on the CRP molecules; or (ii) at least one of the ligands is capable of being bound by a ligand binding site present on a CRP molecule, and at least one other of the ligands is capable of being bound by a ligand binding site present on a serum amyloid P component (SAP) molecule. Suitable ligands for CRP are bis(phosphocholine) ligands, and an exemplified compound, designated BPC8, has the following formula (BPC8):

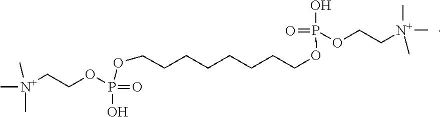

The number 8 in BPC8 refers to the n-octyl linker group in the above formula. Corresponding compounds BPC6, BPC7, etc. having n-hexyl, n-heptyl, etc. linker groups are also disclosed. BPC6 and BPC8 are avidly bound by CRP, cross linking pairs of the native pentameric protein molecules. They completely abrogate the adverse effects of human CRP in the rat acute myocardial infarction model (4, and Pepys et al. unpublished observations). However, the bis(phosphocholine)alkane series of compounds were difficult to synthesise and purify at scale.

International patent application PCT/EP2021/054072 describes an agent for use in medicine, wherein the agent comprises bis-quinuclidinyl compounds having the following formula:

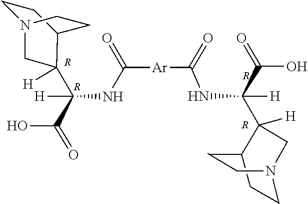

wherein Ar is an aryl linker group such as 1,4-phenyl. These bis-quinuclidinyl compounds are effective inhibitors of human CRP. However, under certain conditions the compounds are thought to exist in zwitterionic forms that may not be sufficiently soluble for some forms of administration.

A need therefore remains for agents or compounds for use in the treatment of medical conditions which are exacerbated by CRP.

### SUMMARY OF THE INVENTION

In a first aspect, the present invention provides an agent for use in medicine, wherein the agent comprises a compound of Formula (I):

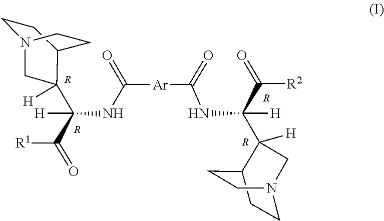

wherein: Ar is an aryl linker group, and
R¹ and R² are independently selected from groups of formula —OH; —OR³;
—O(CO)R³; —NR³R^{3′};

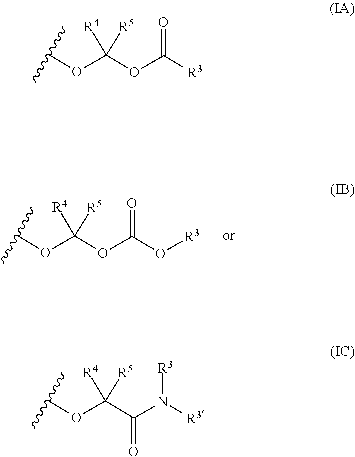

wherein:
R³ and R^{3′} are independently selected from H, C1-C12 alkyl, C1-C12 alkylene, C3-C8 (hetero)cycloalkyl, or C5-C12 (hetero)aryl, or R³ and R^{3′} together form a C3-C8 heterocyclic ring with the nitrogen, each optionally substituted with one, two or three groups selected from halogen, —CF₃, —OR⁶, —NR⁶R^{6′}, —COR⁶, and —CO₂R⁶, C3-C8 (hetero)cycloalkyl, or C5-C12 (hetero)aryl;
R⁴ and R⁵ are independently selected from H or C1-C6 alkyl; and
R⁶ and R^{6′} are independently C1-C6 alkyl, or together form a C3-C8 heterocyclic ring with the nitrogen,
provided that R¹ and R² are not both —OH,
including individual pharmaceutically acceptable salts, solvates, or derivatives thereof.

Suitably, the compound of Formula (I) reacts in vivo in the mammalian body to release a compound of Formula (III):

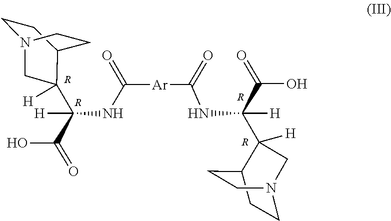

wherein Ar is an aryl linker group as defined above. (The above Formula is understood to include corresponding cations, anions or zwitterions thereof that may be present at physiological pH). Thus, the compounds in the agents of the present invention are prodrugs for the bis-quinuclidinyl compounds of Formula (III).

Suitably, the compound of Formula (III) is an inhibitor of ligand binding by human C-reactive protein (CRP).

In a second aspect, the present invention provides an agent according to the first aspect of the invention, for use in the treatment or prevention of tissue damage in a subject having an inflammatory and/or tissue damaging condition. In a further aspect, the invention provides a pharmaceutical composition comprising an agent according to the first aspect of the invention in admixture with one or more pharmaceutically acceptable excipients, diluents or carriers.

### DETAILED DESCRIPTION OF THE INVENTION

In a first aspect, the present invention provides an agent for use in medicine, wherein the agent comprises a compound of Formula (I):

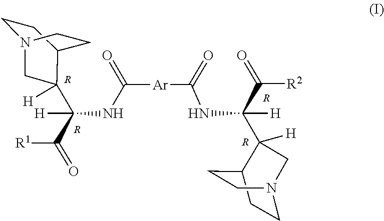

wherein: Ar is an aryl linker group, and
R¹ and R² are independently selected from groups of formula —OH; —OR³;
—O(CO)R³; —NR³R^{3′};

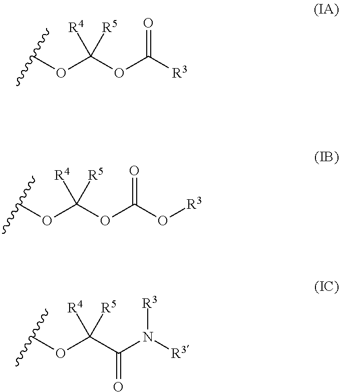

wherein:
R³ and R^{3′} are independently selected from H, C1-C12 alkyl, C1-C12 alkylene, C3-C8 (hetero)cycloalkyl, or C5-C12 (hetero)aryl, or R³ and R^{3′} together form a C3-C8 heterocyclic ring with the nitrogen, each optionally substituted with one, two or three groups selected from halogen, —CF₃, —OR⁶, —NR⁶R^{6′}, —COR⁶, and —CO₂R⁶, C3-C8 (hetero)cycloalkyl, or C5-C12 (hetero)aryl;
R⁴ and R⁵ are independently selected from H or C1-C6 alkyl; and
R⁶ and R^{6′} are independently C1-C6 alkyl, or together form a C3-C8 heterocyclic ring with the nitrogen,
provided that R¹ and R² are not both —OH,
including individual pharmaceutically acceptable salts, solvates, or derivatives thereof.

The Ar linker group is suitably a monocyclic, bicyclic, or fused bicyclic aryl group optionally containing 1, 2 or 3 hetero atoms in the aromatic ring(s), the hetero atoms suitably being selected from N or S. The Ar linker group suitably contains from 4 to 12 carbon atoms in the aromatic rings (i.e. excluding carbon atoms in optional substituent groups). The aromatic ring(s) of the Ar group are linked to the palindromic end groups of the compounds of Formula (I) through amide bonds as shown in Formula (I). Suitably, the bond angle between the two Ar—CO bonds is about 180 degrees. Thus, for example, where Ar is a single six-membered aromatic ring such as a phenyl group, the bonds are suitably located para (1,4) on the ring. It appears that the resulting conformational relationship positions the quinuclidinyl end groups appropriately for binding to respective receptors in the CRP.

In embodiments, the Ar group is selected from 1,4-phenyl, 2,6-naphthyl or 4,4′-biphenyl, or groups of the same ring system containing 1, 2 or 3 heteroatoms in the ring(s), (e.g. 2,6-pyridyl instead of 1,4-phenyl). In each case, the aromatic rings may be substituted with one or more substituent groups R as defined below.

In these embodiments, the linker group Ar may be selected from the group consisting of the following general Formulae Ar-I to Ar-VI:

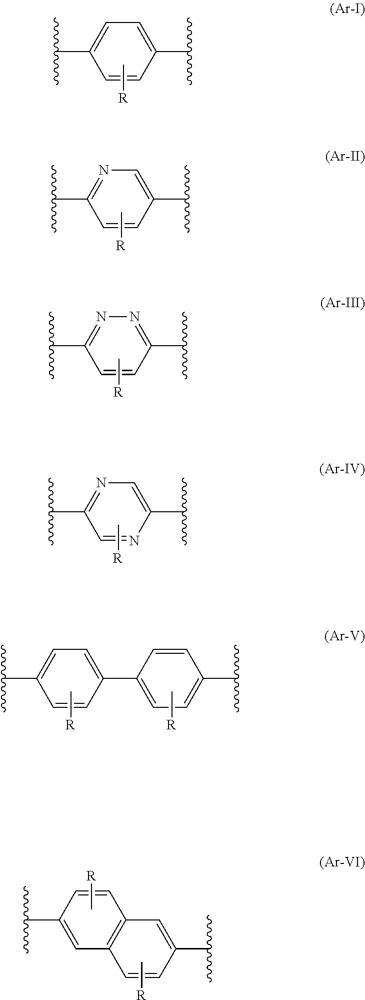

wherein R represents one or more optional substituents on the aryl ring(s). Suitably, R may be selected from halogen, hydroxy, cyano, —CONH₂, or C1-C5 (cyclo)alkyl or C1-C5 (cyclo)alkoxy wherein the alkyl groups are optionally substituted with a phenyl group (e.g. wherein R is —O-benzyl) or with one or more halogen atoms, for example trifluoromethyl. More suitably, R may be C1-C4 alkyl or C1-C4 alkoxy, for example methyl. Suitably, there are 0, 1 or 2 R substituents on the aryl linker, more suitably 0 or 1 R substituents, and in some cases no R substituents. In specific embodiments, the Ar linker group is a 1,4-phenyl linker group having 0, 1 or 2 R substituents.

In embodiments, the aryl linker group Ar is selected from the group consisting of groups having formulae Ar-VII to Ar-XVI:

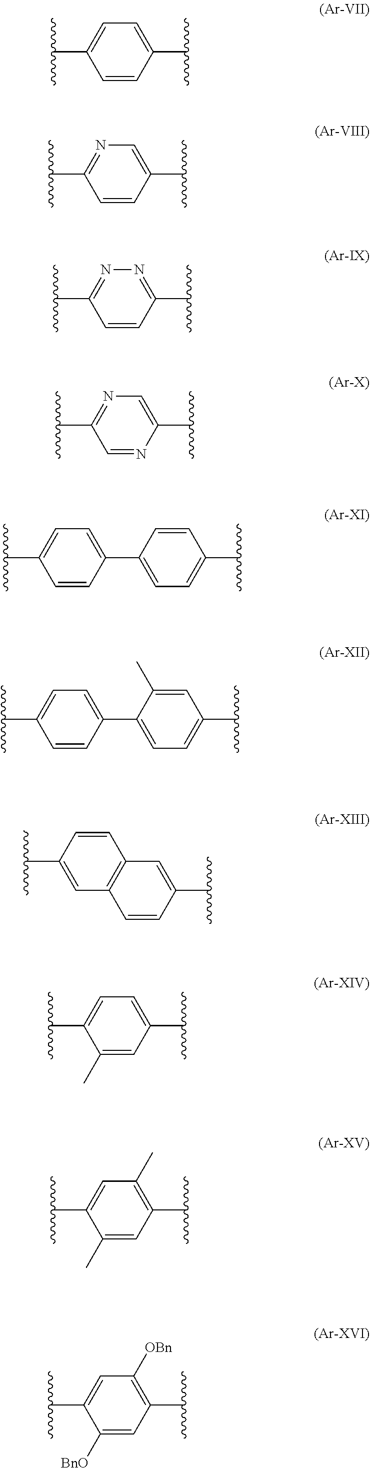

In embodiments of particular interest, the compound of Formula (I) has the following Formula (II):

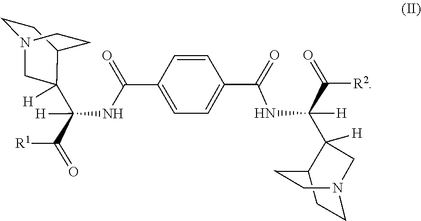

The definitions of Formula (I) and (III) encompass all pharmaceutically acceptable salts, crystalline forms, solvates, and polymorphs of the said compounds.

The compound of Formula (I) is a prodrug compound. The term “prodrug” refers to a derivative of an active compound (drug) that undergoes a transformation under the conditions of use, such as within the body, to release an active drug. The prodrugs of the invention are suitably, but not necessarily, pharmacologically inactive until converted into the active drug. The prodrugs of the invention comprise a promoiety (R¹ and/or R²) protecting carboxyl groups of a drug.

The promoiety is cleavable under specified conditions of use to release the drug having free carboxylate groups. The bond(s) between the drug and promoiety may be cleaved by enzymatic or non-enzymatic means. Under the conditions of use, for example following administration to a subject, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a subject to which the prodrug is administered or the acidic conditions of the stomach or the agent may be supplied exogenously.

Suitably, the compound of Formula (I) reacts in vivo in the mammalian body to release an agent of Formula (III):

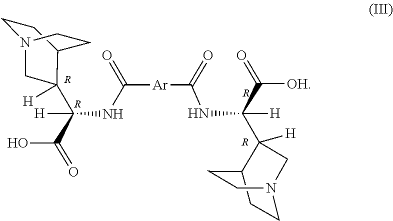

The prodrugs of Formula (I) are predicted on the basis of c Log P calculations to have better solubility than the corresponding drug compounds of Formula (III).

Suitably, the prodrugs of Formula (I) have solubilities in one or more of the following ranges:
(A) In Simulated Gastric Fluid pH 1.2 [SGF], at least about 1000 μg/mL, preferably at least about 2000 μg/mL;
(B) In Simulated Fasted State Intestinal Fluid pH 6.5 [FaSSIF], at least about 1000 μg/mL, preferably at least about 2000 μg/mL; and/or
(C) In Phosphate Buffered Saline pH 7.4 [PBS], at least about 500 μg/mL, preferably at least about 1000 μg/mL.

Suitably, the prodrugs of Formula (I) have c Log P greater than about 0, for example from about 0 to about 7, suitably from about 0 to about 5. The c Log P values may be calculated using Chemdraw® software.

Additionally or alternatively, the prodrugs of Formula (I) may provide one or more of the following advantages relative to the drugs of Formula (III):
(1) decreased toxicity and/or decreased adverse reactions such as gastric irritation;
(2) improved pharmacokinetics;
(3) improved stability;
(4) improved crystallinity;
(5) improved ease of isolation and/or purification; and/or
(6) improved organoleptic properties.

The groups R¹ and R² in Formula (I) may be any suitable groups that are converted to —OH groups by reaction, suitably by hydrolysis, in vivo in the mammalian body. Preferably, in the compounds of Formula (I), neither R¹ nor R² is —OH. Preferably, R¹ and R² are the same, which may result in a palindromic compound of Formula (I) where the group Ar is palindromic (e.g. when Ar is 1,4-phenylene). It will be appreciated that it is easier to synthesise and characterize compounds having identical R¹ and R².

R³ and R^{3′} are independently selected from H, C1-C6 alkyl, C1-C6 alkylene, C3-C8 (hetero)cycloalkyl, or C5-C12 (hetero)aryl, or R³ and R^{3′} together form a C3-C8 heterocyclic ring with the nitrogen to which the R³ and R^{3′} groups are attached.

The above R³ and R^{3′} groups may each optionally be substituted with one, two or three groups selected from halogen, —CF₃, —OR⁶, —NR⁶R^{6′}, —COR⁶, and —CO₂R⁶, C3-C8 (hetero)cycloalkyl, or C5-C12 (hetero)aryl, wherein R⁶ and R^{6′} are independently C1-C6 alkyl, or together form a C3-C8 heterocyclic ring with the nitrogen to which R⁶ and R^{6′} are attached.

The alkyl and alkylene groups herein may be straight or branched.

The term “(hetero) cycloalkyl” refers to a cycloalkyl group optionally having one or more —O—, —S—, —C(O)—, or —NH— groups in the ring, including fused ring structures, bicyclic ring structures, and non-aromatic unsaturated ring structures. The term “(hetero) cycloalkyl” thereby encompasses cyclic ketones, lactones, cyclic carbonates, and cyclic anhydrides The term “(hetero)aryl” refers to an aryl group optionally having one or more —O—, —S— or —N— groups in the ring, including fused aromatic ring structures.

Suitably, the groups R¹ and/or R² have relatively low molecular weight in order to minimise steric hindrance to hydrolysis of the prodrug, and to maximise the fraction of the pharmaceutically effective material in the agent. Thus, for example, the groups R¹ and/or R² each suitably have a molecular weight of 200 or less, in embodiments 150 or less, typically 100 or less.

In embodiments, the groups R¹ and/or R² are —OR³ groups, thereby forming simple ester terminal group(s) on the compound of Formula (I). In these embodiments, suitably the groups R¹ and R² are identical C1-C6 saturated and unsubstituted oxoalkyl groups, in particular —OMe, —OEt, or —OiPr.

In alternative embodiments, the groups R¹ and/or R² are —O(CO)R³ groups, thereby forming anhydride terminal group(s) on the compound of Formula (I), wherein the R³ groups are as defined above. In these embodiments, R³ may suitably be methyl, whereby hydrolysis of the anhydride releases acetate and the drug of formula (III).

In alternative embodiments, the groups R¹ and/or R² are —NR³R^{3′} groups, thereby forming amide terminal group(s) on the compound of Formula (I).

In alternative embodiments, the groups R¹ and/or R² are groups of Formula (IA):

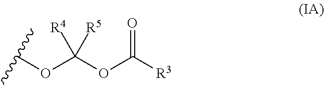

wherein the R³ group is as defined above, thereby forming acyloxymethyl ester end groups on the prodrugs of Formula (I). In these embodiments, suitably R⁴ is H and R⁵ is H or C1-C3 alkyl, more suitably methyl. In these embodiments, suitably R³ is methyl, ethyl, or isopropyl, or R³ is 4-tetrahydropyranyl.

In alternative embodiments, the groups R¹ and/or R² are groups of Formula (IB):

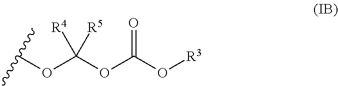

wherein the R³ group is as defined above, thereby forming alkylcarbonate ester end groups on the prodrugs of Formula (I). In these embodiments, suitably R⁴ is H and R⁵ is H or C1-C3 alkyl, more suitably methyl. In these embodiments, suitably R³ is methyl, ethyl, isopropyl, or more suitably hydroxymethyl.

In alternative embodiments, the groups R¹ and/or R² are groups of Formula (IC):

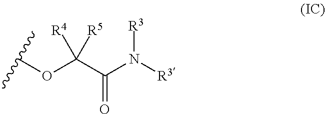

wherein both the R³ group and the R^{3′} group are as defined above for R³ and R^{3′}. In these embodiments, suitably R⁴ is H and R⁵ is H or C1-C3 alkyl, more suitably methyl.

In all cases, it is expected that the prodrugs of the invention will have improved solubility, permeability, and therefore bioavailability than the zwitterionic compounds of Formula (III). Chemdraw© was used to obtain the following calculated log P values (c Log P) for selected compounds of the invention having identical groups R¹ and R²:

R¹, R²=—OMe, c Log P=0.33; R¹, R²=OEt, c Log P=1.0; R¹, R²=OiPr, c Log P=1.64; R¹, R²=OtBu, c Log P=2.08; R¹, R²=—OCH₂OC(O)Me, c Log P=0.3; R¹, R²=—OCH₂OC(O)Et, c Log P=1.61; R¹, R²=—OCH₂OC(O)iPr, c Log P=2.25; R¹, R²=—OCH₂OC(O)tBu, c Log P=2.89; R¹, R²=—OCH₂OC(O)OMe, c Log P=1.48; R¹, R²=—OCH₂OC(O)OEt, c Log P=2.15; R¹, R²=—OCH₂OC(O)OiPr, c Log P=2.79; R¹, R²=—OCH₂OC(O)OtBu, c Log P=3.43; R¹, R²=—OCH₂(3-citraconic anhydride), c Log P=−0.36.

The specific choice of groups R¹ and R² may also be determined by other factors. For example, it is to be expected that the anhydride or alkylcarbonate ester prodrug groups may undergo more rapid hydrolysis to the prodrug in vivo, which may be desirable. It is to be expected that prodrugs that release only innocuous substances such as acetate together with the drug of Formula (III) upon hydrolysis will be preferable. Alternatively, simple ester prodrugs may be primarily hydrolysed by esterase enzymes, in particular in the liver, which may provide more desired pharmacokinetics.

It has been found that the above bivalent ligand compounds of Formula (III) that are generated in vivo in the mammalian body from the prodrugs of Formula (I) are avidly bound by human CRP in vitro and in vivo, forming stable complexes of pairs of native pentameric CRP molecules cross-linked by up to 5 ligand molecules. The ligand binding pockets of each CRP protomer are blocked, and the whole binding (B) face of each CRP pentamer is fully occluded in this complex so that CRP cannot mediate tissue damaging action in vivo. Furthermore, dissociation of the individual, non-covalently associated, protomers of native CRP from within the CRP-ligand complex is completely inhibited under physiological conditions.

Suitably, the compound of Formula (III) is an inhibitor of human C-reactive protein (CRP) having an IC₅₀ of about 20 μM or less, suitably about 10 μM or less, more suitably about 5 μM or less, or most suitably about 1 μM or less. A suitable method of measuring the IC₅₀ to determine whether a particular compound has IC₅₀ within the specified ranges is described herein below.

The compounds of Formulas (I), (II) and (III) are R,R,R,R stereoisomers. The other stereoisomers of drugs according to Formula (III) derived from the compounds of Formula (I) and (II) have been found to have lesser activity. The S,S,S,S isomer is thought to be the most active alternative stereoisomer.

Suitably, the diastereomeric purity of the (R,R,R,R) stereoisomer in the agents of the invention is at least about 50% by weight, suitably at least about 60%, more suitably at least about 75%, still more suitably at least about 90%, and most suitably at least about 98%. That is to say, the amount of the (R,R,R,R) stereoisomer suitably exceeds the amount of all other stereoisomers of this compound present in the agent. Most suitably, at least about 98% by weight of all stereoisomers of this compound present in the agent is the R,R,R,R stereoisomer.

In a second aspect, the present invention provides an agent according to the invention for use in the treatment or prevention of a medical condition mediated by CRP. In another aspect, the present invention provides the use of an agent according to the first aspect of the invention for the manufacture of a medicament for treatment or prevention of a medical condition mediated by CRP.

The agents according to the invention, comprising the compound of Formula (I), may be administered concurrently with one or more other pharmaceutically active medications, simultaneously, separately or sequentially. Such other pharmaceutically active medications may include, for example, anti-inflammatory drugs such as corticosteroids; anti-viral, anti-bacterial, anti-fungal or anti-parasitic drugs; inhibitors/antagonists of pro-inflammatory cytokines such as IL-1, IL-6, TNF; anti-coagulants; inhibitors of complement activation or its bioactive fragments.

The present invention further provides a method for treating a medical condition mediated by CRP in a patient in need thereof, comprising administering to the patient a therapeutic amount of an agent according to the invention, or a pharmaceutical composition according to the invention.

In embodiments, the inflammatory and/or tissue damaging condition comprises one or more of acute coronary syndrome, unstable angina, plaque rupture, and/or incipient atherothrombosis.

In embodiments, the inflammatory and/or tissue damaging condition is selected from an infection, an allergic complication of infection, an inflammatory disease, ischemic or other necrosis, traumatic tissue damage and malignant neoplasia. For example, the condition may be an infection selected from a bacterial infection including sepsis, a viral infection, a fungal infection and a parasitic infection.

In embodiments, the condition is an inflammatory disease selected from rheumatoid arthritis, juvenile chronic (rheumatoid) arthritis, ankylosing spondylitis, psoriatic arthritis, systemic vasculitis, polymyalgia rheumatica, Reiter's disease, Crohn's disease and familial Mediterranean fever and other autoinflammatory conditions.

In embodiments, the condition is tissue necrosis selected from myocardial infarction, ischaemic stroke, tumour embolization and acute pancreatitis.

In embodiments, the condition is trauma selected from elective surgery, burns, chemical injury, fractures and compression injury.

In embodiments, the condition is malignant neoplasia selected from lymphoma, Hodgkin's disease, carcinoma and sarcoma.

In embodiments, the condition is an allergic complication of infection selected from rheumatic fever, glomerulonephritis, and erythema nodosum leprosum.

In embodiments, the condition is an infection or complication of infection with a severe acute respiratory syndrome (SARS) coronavirus, in particular SARS-CoV2 or Covid-19.

Suitably, the method involves administering to a patient an amount of the agent according to the invention sufficient to bind all CRP in the circulation and extracellular tissue fluids. For example, the amount may be sufficient to bind at least about 70% of the available CRP, preferably at least about 90% of available CRP and optimally 95%, 99% or 100% of the available CRP.

In a further aspect, the present invention provides a pharmaceutical composition comprising an agent according to the first aspect of the invention in admixture with one or more pharmaceutically acceptable excipients, diluents or carriers.

Pharmaceutical compositions may be formulated comprising an agent or a pharmaceutically acceptable salt, ester or prodrug thereof according to the present invention optionally incorporating a pharmaceutically acceptable carrier, diluent or excipient (including combinations thereof). Here and elsewhere in the present specification, the term “pharmaceutically acceptable salt” refers to salts of the compounds of Formula (I) with anions or cations of which are known and accepted in the art for the formation of salts for pharmaceutical use. Acid addition salts, for example, may be formed by mixing a solution of the agent with a solution of a pharmaceutically acceptable, non-toxic acids, which include but are not limited to hydrochloric acid, oxalic acid, fumaric acid, maleic acid, succinic acid, acetic acid, citric acid, tartaric acid, carbonic acid or phosphoric acid. Where the agent carries a carboxylic acid group, the invention also contemplates salts thereof, preferably non-toxic, pharmaceutically acceptable salts thereof, which include, but are not limited to the sodium, potassium, calcium and quaternary ammonium salts thereof.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Antioxidants and suspending agents may be also used.

The pharmaceutical compositions may be in the form of a prodrug comprising the agent or a derivative thereof which becomes active only when metabolised by the recipient. The exact nature and quantities of the components of such pharmaceutical compositions may be determined empirically and will depend in part upon the route of administration of the composition. Where appropriate, the pharmaceutical compositions of the present invention can be administered by inhalation, in the form of a suppository or pessary, topically (including ophthalmically) in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly, subcutaneously or intra-arterially.

The liquid forms in which the compositions of the present invention may be incorporated for administration by injection include aqueous emulsions with edible oils such as cottonseed oil, sesame oil, coconut oil and peanut oil, as well as elixirs and similar pharmaceutical vehicles. Suitable dispersing or suspending agents for aqueous suspension include synthetic and natural gums such as tragacanth, acacia, alginate, dextran, sodium carboxymethylcellulose, methylcellulose, polyvinyl-pyrrolidone and gelatin.

For parenteral administration, the compositions may be best used in the form of a sterile aqueous solution which may contain other substances, for example buffers to adjust pH, or enough salts or monosaccharides to make the solution isotonic with blood. For buccal or sublingual administration the compositions may be administered in the form of tablets or lozenges which can be formulated in a conventional manner.

Use of the compounds of the present invention aims to saturate with the ligand drug all circulating and other extracellular CRP molecules in the body. The daily dose of drug required is therefore suitably that which provides at least about 1 mol of drug, more suitably at least about 5 mol of drug per mol of native pentameric CRP to be complexed.

The precise form of pharmaceutical composition and dosage thereof may also be dependent on the subject to be treated, including body weight, route of administration and disease conditions. These would be determined as a matter of routine by the skilled addressee.

The prodrugs of Formula (I) may be made from the drug compounds of Formula (III) by suitable chemical reactions known to those skilled in the art.

For example, where the groups R¹ and R² are —OR³ groups, thereby forming ester terminal groups on the compound of Formula (I), conventional esterification methods as known in the art or described herein may be used. Methods of making ester prodrugs are described, for example, in WO2014/078669, WO2007/127639 and WO03/051836. Ester prodrugs can provide additional advantages of stability in gastric or intestinal fluids (see below), with release of the drug taking place after absorption into the blood and tissues such as the liver by the action of esterase enzymes.

In embodiments where the groups R¹ and/or R² are —O(CO)R³ groups, thereby forming anhydride terminal group(s) on the compound of Formula (I), the prodrugs may for example be prepared by the methods described in WO91/09831. Anhydride prodrugs can provide advantages of relatively rapid release of the prodrug by hydrolysis in the plasma.

In embodiments where the groups R¹ and/or R² are —NR³R^{3′} groups, thereby forming amide terminal group(s) on the compound of Formula (I), the prodrugs may be prepared by any suitable method of amide synthesis.

In embodiments where the groups R¹ and/or R² are groups of Formula (IA):

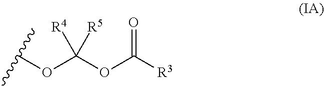

thereby forming acyloxymethyl ester end groups on the prodrugs of Formula (I), the prodrugs may for example be prepared by the methods described in WO2017/072099 and WO03/051836. The acyloxymethyl ester end groups can provide advantages of improved crystallinity and enhanced hydrolysis rates compared to simple esters. In addition, the hydrolysis products of the acyloxymethyl esters (besides the drug itself) can be innocuous.

In embodiments, the groups R¹ and/or R² are groups of Formula (IB):

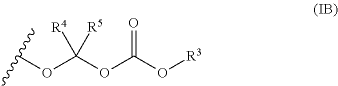

wherein the R³ group is as defined above, thereby forming alkylcarbonato oxymethyl ester end groups on the prodrugs of Formula (I). The acyloxymethyl ester end groups can provide advantages of improved crystallinity and enhanced hydrolysis rates compared to simple esters. In addition, the hydrolysis products of the acyloxymethyl esters (besides the drug itself) can be innocuous.

In embodiments where the groups R¹ and/or R² are groups of Formula (IC):

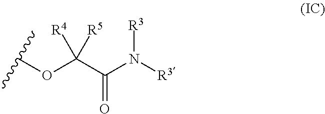

wherein both the R³ group and the R^{3′} group are as defined above, resulting in carbamoyl methyl ester prodrugs. These prodrugs may for example be prepared by the methods described in WO88/01615. The acyloxymethyl ester end groups can provide advantages of improved crystallinity and enhanced hydrolysis rates compared to simple esters. These prodrugs are rapidly hydrolysed by plasma enzymes, which may be preferable to hydrolysis by liver esterase enzymes.

Alternatively, the compound of Formula (I) may be made by reacting a compound of Formula (IV):

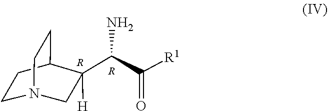

wherein R¹ is as defined above, and is effective as a carboxyl protecting group, with a compound of Formula (IV-A) or (IV-B):

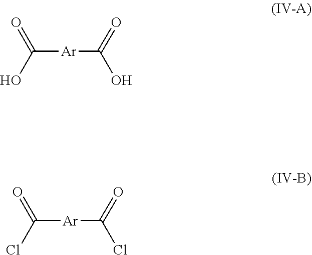

to form a compound of Formula (I) wherein R¹ and R² are the same.

The step of reacting the compound of Formula (IV) with the compound of Formula (IV-A) to form the compound of Formula (I) may be performed by any of the methods conventionally used to form amide bonds in peptide synthesis. For example, the —COOH groups of the compound of Formula (IV-A) may be activated by converting them into esters of strong acids or groups of formula —COX, where X is a leaving group that is readily displaced by nucleophilic substitution such as chloro, alkylsulfonate or toluenesulfonate, followed by nucleophilic reaction with the primary amine groups of the compounds of Formula (IV). In other embodiments, activation of the carboxylic acid may be performed with either phosphate containing reagents, triazine based reagents, carbodiimide based reagents or hydroxybenzotriazole based reagents in the presence of an organic solvent and base. Preferred conditions comprise TBTU (2-(1H-benzotriazole-1-yl)-1,1,3,3-tetramethylaminium tetrafluoroborate with diisopropylethylamine in MeCN at room temperature.

Alternatively, compounds of Formula (I) may be prepared using the bis acid chloride of compound (IV-B). Typical reaction conditions comprise warming to 30° C. in chloroform for 16 hours.

Compounds of Formula (IV) and (IV-A/B) are either commercially available, prepared according to the methods described herein, or prepared according to the literature.

For example, the compound of Formula (IV) may be made by a method comprising the step of reacting a compound of Formula (VI) with a compound of Formula (VII)

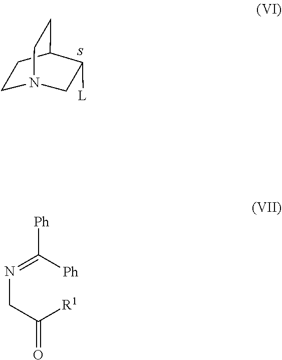

wherein L represents a leaving group, i.e. a weakly basic group that is readily displaced by nucleophilic substitution. Suitably leaving groups L include bromo, iodo, alkylsulfonate and phenylsulfonate groups, such as a p-bromophenylsulfonate. R¹ is a carboxyl protecting group as defined above. The reaction is suitably carried out in the presence of a strong non-nucleophilic base in an aprotic solvent. For example, the strong base may be potassium bis(trimethylsilyl) amide, KHMDS and the solvent may be toluene/THF. The reaction proceeds by nucleophilic substitution to form a mixture of stereoisomers of Formula (VIII-A) and (VIII-B):

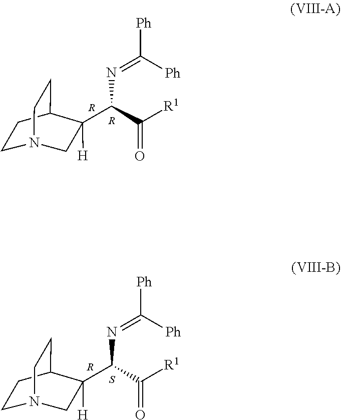

The synthesis of the compound of Formula (IV) then comprises hydrolysing and resolving the above mixture of stereoisomers to isolate the compound of Formula (IV) or a salt thereof with an optically active organic acid compound. The hydrolysis may be performed with H₂O under mild acidic conditions, for example in the presence of (1S)-10-camphorsulfonic acid. The salt of Formula (IV). 2CSA is preferentially precipitated from the mixture. Other chiral organic acids that are commonly used for separating enantiomers may be suitable, for example (2S,3S)-tartaric acid, (R)-Malic acid, or (−)-(R)-mandelic acid.

### EXAMPLES

The invention will now be illustrated, but not limited, by reference to the specific embodiments described in the following examples. Compounds are named using conventional IUPAC nomenclature, or as named by the chemical supplier.

The following synthetic procedures are provided for illustration of the methods used; for a given preparation or step the precursor used may not necessarily derive from the individual batch synthesized according to the step in the description given.

### Analytical Methods

Where examples and preparations cite analytical data, one of the following analytical methods were used unless otherwise specified.

NMR: 400 MHz Bruker Avance III and Bruker Avance Neo.

### LC-MS or HPLC Method:

### Method 1:

MS instrument type: SHIMADZU LC-MS-2020, Column: Kinetex EVO C18 30×2.1 mm, 5 μm, mobile phase A: 0.0375% TFA in water (v/v), B: 0.01875% TFA in Acetonitrile (v/v), gradient: 0.0 min 0% B→0.8 min 60% B→1.20 min 60% B→1.21 min 0% B→1.55 min 0% B flow rate: 1.5 mL/min, oven temperature: 50° C.; PDA detection: 220 nm & 254 nm.

### Method 2:

MS instrument type: Agilent 1200 LC/G1956A MSD, Column: Kinetex EVO C18 2.1×30 mm, 5 um, mobile phase A: 0.0375% TFA in water (v/v), B: 0.01875% TFA in Acetonitrile (v/v), gradient: 0.0 min 90% B→0.35 min 90% B flow rate: 1.5 mL/min, oven temperature: 50° C.; DAD: 100-1000.

### Method 3:

HPLC instrument type: SHIMADZU LC-20AB, Column: Kinetex C18 LC Column 4.6×50 mm, 5 μm, mobile phase A: 0.0375% TFA in water (v/v), B: 0.01875% TFA in Acetonitrile (v/v), gradient: 0.0 min 0% B→4.20 min 60% B→5.30 min 60% B→5.31 min 0% B→6.00 min 0% B, flow rate: 1.5 mL/min, oven temperature: 50° C.; PDA detection: PDA (220 nm&215 nm&254 nm).

### Method 4:

MS instrument type: SHIMADZU LC-MS-2020, Column: Kinetex EVO C18 30×2.1 mm, 5 μm, mobile phase A: 0.0375% TFA in water (v/v), B: 0.01875% TFA in Acetonitrile (v/v), gradient: 0.0 min 0% B→3.0 min 60% B→3.50 min 60% B→3.51 min 0% B→4.00 min 0% B flow rate: 0.8 mL/min, oven temperature: 50° C.; PDA detection: 220 nm & 254 nm.

### Method 5:

MS instrument type: SHIMADZU LC-MS-2020, Column: Kinetex EVO C18 2.1×30 mm, 5 μm, mobile phase A: 0.025% NH3·H2O in Water (v/v), B: Acetonitrile, gradient: 0.0 min 0% B→0.8 min 60% B→1.20 min 60% B→1.21 min 0% B→1.55 min 0% B flow rate: 1.5 mL/min, oven temperature: 40° C.; PDA detection: 220 nm & 254 nm.

### Method 6:

HPLC instrument type: SHIMADZU LC-20AB, Column: Kinetex C18 LC Column 4.6×50 mm, 5 μm, mobile phase A: 0.0375% TFA in water (v/v), B: 0.01875% TFA in Acetonitrile (v/v), gradient: 0.0 min 0% B→4.20 min 30% B→5.30 min 30% B→5.31 min 0% B→6.00 min 0% B, flow rate: 1.5 mL/min, oven temperature: 50° C.; PDA detection: PDA (220 nm&215 nm&254 nm).

### Method 7:

MS instrument type: SHIMADZU LC-20AB, Column: Kinetex C18 LC Column 4.6×50 mm, 5 μm, mobile phase A: 0.0375% TFA in water (v/v), B: 0.01875% TFA in Acetonitrile (v/v), gradient: 0.0 min 0% B→2.40 min 30% B→3.70 min 30% B→3.71 min 0% B→4.00 min 0% B flow rate: 1 mL/min, oven temperature: 50° C.; PDA detection: 220 nm & 254 nm.

### Method 8:

MS instrument type: Agilent 1100 LC & Agilent G1956A, Column: Waters XSelect HSS T3 3.5 μm 4.6×50 mm, mobile phase A: 0.0375% TFA in water (v/v), B: 0.01875% TFA in Acetonitrile (v/v), gradient: 0.0 min 0% B→5.00 min 30% B→6.00 min 100% B→6.50 min 100% B→6.51 min 0% B→7.00 min 0% B flow rate: 1 mL/min, oven temperature: 40° C.; PDA detection: 220 nm & 254 nm.

### Method 9

MS instrument type: SHIMADZU LCMS-2020, Column: Kinetex EVO C18 2.1×30 mm, 5 μm, mobile phase A: 0.025% NH₃·H₂O in Water (v/v), B: Acetonitrile, gradient: 0.0 mins 5% B→0.8 mins 95% B→1.2 mins 95% B→1.21 mins 5% B→1.55 mins 5% B, flow rate: 1.5 mL/mins, oven temperature: 40° C.; UV detection: 220 nm & 254 nm.

### Method 10

MS instrument type: Agilent 1100 LC & Agilent G1956A, Column: K Waters XSelect HSS T3 3.5 μm 4.6×50 mm, mobile phase A: 0.0375% TFA in water (v/v), B: 0.01875% TFA in Acetonitrile (v/v), gradient: 0.0 mins 0% B→5 mins 30% B→6 mins 100% B→6.5 mins 100% B→6.51 mins 0% B, flow rate: 0.6 mL/mins, oven temperature: 40° C.; UV detection: 220 nm & 254 nm.

### HPLC Method 1:

MS instrument type: SHIMADZU LC-20AB, Column: XBridge® C18 3.5 μm 4.6×150 mm, mobile phase A: 0.0375% TFA in water (v/v), B: 0.01875% TFA in Acetonitrile (v/v), gradient: 0.0 mins 0% B→10.0 mins 60% B→15.0 mins 60% B→15.01 mins 0% B→15.02 mins 0% B→20.0 mins 0% B, flow rate: 1.0 mL/mins, oven temperature: 40° C.; UV detection: 220 nm &215 nm & 254 nm.

### Abbreviations

Where the following abbreviations have been used, the following meanings apply:
ACN or MeCN is acetonitrile,
CDCl₃ is deuterochloroform,
CSA is Camphor-10-sulfonic acid,
D₂O is deuterium oxide,
DCM is dichloromethane,
DIPEA or DIEA is N,N-diisoproylethylamine,
DMAP is 4-(dimethylamino)pyridine,
DMSO is dimethyl sulfoxide,
EA is ethyl acetate,
EtOH is ethanol,
FA is formic acid,
H₂O is water,
HCl is hydrochloric acid,
HPLC is high performance liquid chromatography,
IPA is isopropyl alcohol,
KHMDS is potassium bis (trimethylsilyl)amide,
KOH is potassium hydroxide,
LCMS is liquid chromatography mass spectrometry,
MeOH is methanol,
MTBE is methyl tert butyl ether,
N₂ is nitrogen,
Na₂SO₄ is sodium sulfate;
NH₃ is ammonia,
NH₄HCO₃ is ammonium bicarbonate,
NMR is nuclear magnetic resonance,
PDA is photodiode array detector,
SFC is supercritical fluid chromatography,
TBTU is 2-(1H-Benzotriazole-1-yl)-1,1,3,3-tetramethylaminium tetrafluoroborate,
TEA is triethylamine,
TFA is trifluoroacetic acid,
THF is tetrahydrofuran and
TLC is thin layer chromatography.

### PREPARATIONS

### Preparation of compound 2: [(3S)-quinuclidin-3-yl]4-bromobenzenesulfonate

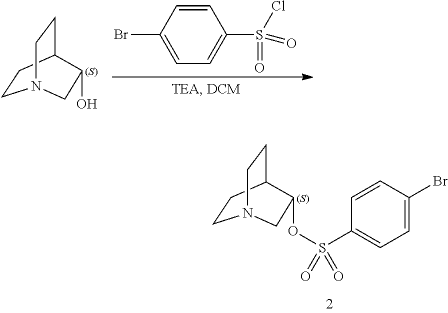

To a solution of (3S)-quinuclidin-3-ol (2.00 g, 15.73 mmol, 1.00 eq), DMAP (19.21 mg, 157.30 μmol, 0.01 eq) and TEA (4.78 g, 47.19 mmol, 6.54 mL, 3.00 eq) in DCM (40.00 mL) was added 4-bromobenzenesulfonyl chloride (6.03 g, 23.60 mmol, 1.50 eq) at 0° C. The mixture was stirred for 16 hours at 20° C. The mixture was washed with sat. NaHCO₃ (100 mL). The sat. NaHCO₃ layer was extracted with EA (100 mL×2). The combined organic layers were dried over Na₂SO₄ and concentrated under reduced pressure to give a crude product as a yellow oil. The yellow oil was purified by chromatography on silica gel eluted with DCM:MeOH=30:1 to give [(3S)-quinuclidin-3-yl]4-bromobenzenesulfonate (3.20 g, 8.32 mmol, 52.88% yield, 90% purity) as a yellow solid, which was analyzed by ¹HNMR.

¹H NMR: (400 MHz, CDCl₃) δ ppm: 7.81-7.57 (m, 4H), 4.65-4.49 (m, 1H), 3.04 (dd, J=8.4, 15.2 Hz, 1H), 2.89-2.48 (m, 5H), 1.93 (d, J=2.8 Hz, 1H), 1.80-1.71 (m, 1H), 1.61 (tdd, J=4.6, 9.5, 13.9 Hz, 1H), 1.47-1.22 (m, 2H).

### Preparation of Diastereomers 4A and 4B

### Isomer 4A: (R)-methyl 2-((diphenylmethylene)amino)-2-((3R)-quinuclidin-3-yl)acetate

### Isomer 4B: (S)-methyl 2-((diphenylmethylene)amino)-2-((3R)-quinuclidin-3-yl)acetate

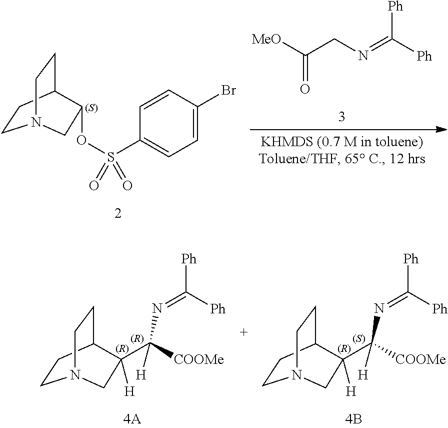

To a solution of (3S)-quinuclidin-3-yl 4-bromobenzenesulfonate (63 g, 182 mmol) and methyl 2-[(diphenylmethylidene)amino]acetate (92.2 g, 364 mmol) in Toluene (578 mL) and THF (186 mL) was added KHMDS (0.70 M in toluene, 520 mL) under N₂ and the reaction was stirred at 65° C. for 12 hours. The reaction mixture was cooled, poured into water (1.00 L) and ethyl acetate (1500 mL) was added. The phases were separated and the aqueous phase was extracted with ethyl acetate (3×1.00 L). The organic layer was washed with saturated brine (2×500 mL), dried over Na₂SO₄, filtered and concentrated in vacuo. The crude mixture (113 g) was obtained as a dark brown oil and used directly in the next step.

Selected NMR data of crude material showed dr (R,R) (R,S)=2.3:1

¹H-NMR: 400 MHz, DMSO-d₆: crude selected δ ppm 4.08 (d, J=8.8 Hz, 2H), 3.95 (d, J=10.2 Hz, 1H).

### Preparation of 5. 2 (+)-CSA Salt (R,R)

### (R)-methyl 2-amino-2-((3R)-quinuclidin-3-yl)acetate bis(((1S,4R)-7,7-dimethyl-2-oxobicyclo[2.2.1]heptan-1-yl)methanesulfonate)

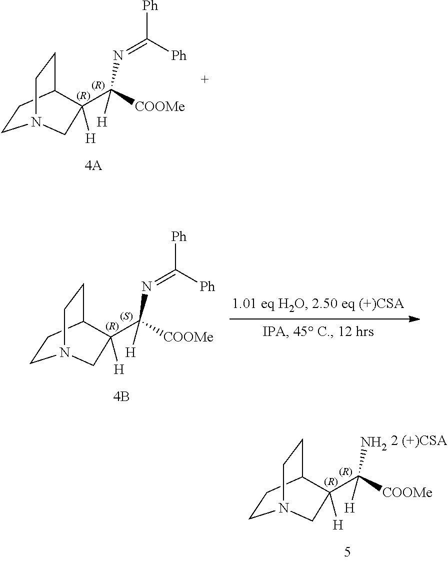

To a solution of the crude reaction mixture of 4A and 4B (105 g, 177 mmol) in IPA (700 mL) was added H₂O (3.21 g, 178 mmol) and the reaction was warmed to 45° C. A solution of (+) CSA (103 g, 442 mmol) in IPA (300 mL) was added and the reaction continued stirring at 45° C. for 12 hrs. The reaction mixture was cooled to 25° C. and filtered to obtain a white solid. The solid was washed with IPA (100 mL) and MTBE (100 mL) and dried under vacuum to obtain the title compound as a white solid (62.0 g, 93.5 mmol, 52.9% yield).

¹H-NMR: 400 MHz, DMSO-d₆: δ ppm 9.62-9.58 (br, s, 1H), 8.51 (br, s, 3H), 4.25-4.22 (d, J=10.4 Hz, 1H), 3.78 (s, 3H), 3.25-3.23 (m, 5H), 2.90-2.86 (d, J=14.8 Hz, 2H), 2.66 (m, 2H), 2.41-2.37 (d, J=14.8 Hz, 3H), 1.95-1.93 (m, 2H), 1.85-1.78 (m, 11H), 1.30-1.27 (m, 4H), 1.04 (s, 6H), 0.74 (s, 6H).

### Confirmation of Stereochemistry for Compound 5. 2 (+)-CSA Salt (R,R)

20 mg compound 4A was dissolved in 1.3 mL dichloromethane/cyclohexane/methanol (5:5:3). The solution was kept in a half sealed 4 mL vial and evaporated slowly at room temperature. Crystals were observed in the second day and a crystal was selected for X-ray crystallographic analysis.

The crystal was a colorless needle with the following dimensions: 0.10×0.02×0.02 mm 3. The symmetry of the crystal structure was assigned the monoclinic space group P2₁ with the following parameters: a=7.0236(2) Å, b=26.8204(6) Å, c=18.0068(5) Å, α=90°, β=99.114(3)°, γ=90°, V=3349.22(16) Å³, Z=4, Dc=1.315 g/cm³, F(000)=1424.0, μ(Cu Kα)=1.918 mm⁻¹ and T=293(2) K using a Rigaku Oxford Diffraction XtaLAB Synergy four-circle diffractometer equipped with a HyPix-6000HE area detector. Cryogenic system: Oxford Cryostream 800 Cu: λ=1.54184 Å, 50 W, Micro focus source with multilayer mirror (μ-CMF). Distance from the crystal to the CCD detector: d=35 mm Tube Voltage: 50 kV Tube Current: 1 mA.

The absolute configuration of 5. 2 (+)-CSA salt was assigned (R,R).

### Preparation of Compound 5 Free Parent from Compound 5 CSA Salt

### (R)-methyl 2-amino-2-((3R)-quinuclidin-3-yl)acetate

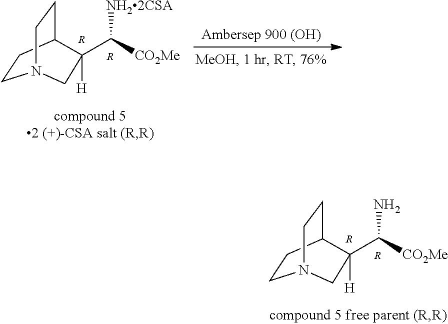

To a suspension of Ambersep 900 (470 g) in MeOH (900 mL) was added methyl (2R)-2-amino-2-[(3R)-1-azabicyclo[2.2.2]octan-3-yl]acetate bis (+) Camphorsulfonic acid salt (Preparation 1, 47.0 g, 70.9 mmol) and the mixture was stirred at 20° C. under N₂ for 1 hour. The reaction mixture was filtered and concentrated in vacuo to afford the title compound (11.0 g, 55.5 mmol, 78.3% yield) as a yellow oil.

¹H-NMR 400 MHz (DMSO-d₆): δ ppm: 7.64-7.34 (m, 8H), 7.25-7.09 (m, 2H), 4.09 (d, J=8.8 Hz, 1H), 3.60 (s, 3H), 2.92-2.77 (m, 1H), 2.73-2.60 (m, 2H), 2.55 (br d, J=6.4 Hz, 1H), 2.43-2.21 (m, 3H), 1.66-1.33 (m, 3H), 1.19 (br d, J=5.2 Hz, 2H).

### Preparation of Compound 6

### (R,2R,2′R)-2,2′-(terephthaloylbis(azanediyl))bis(2-((R)-quinuclidin-3-yl)acetic acid) bis hydrochloride salt

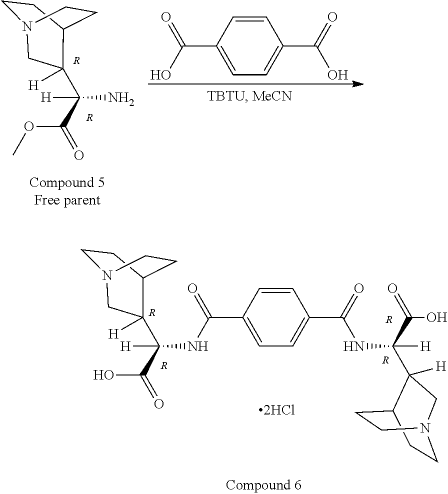

### Step 1

To (R)-methyl 2-amino-2-((3R)-quinuclidin-3-yl)acetate (330 g, 333 mmol, 20% solution in MeCN) and benzene-1,4-dicarboxylic acid (20.50 g, 123 mmol) in MeCN (1.30 L) was added TBTU (88.2 g, 275 mmol) under N₂, followed by DIEA (65.3 g, 505 mmol, 88.0 mL). The reaction was stirred at 25° C. for 12 hrs. The reaction mixture was concentrated in vacuo to afford a crude yellow oil that was taken directly on to the next step.

### Step 2

To a solution of the crude reaction mixture (64.9 g, 123 mmol) in IPA (1.07 L) was added KOH (69.2 g, 123 mmol, 1.07 L, 10% aqueous) and the reaction was stirred at 50° C. under N₂ for 1 hour. The reaction mixture was filtered and the mother liquor was extracted with ethyl acetate (2×300 mL). The aqueous layer was adjusted to pH=4-5 with formic acid and stirred for 12 hours. The resultant white solid was filtered and stirred in water (740 mL) at 90° C. for 2 hours before cooling to 25° C. The solid was filtered, washed with water (2×300 mL) and dried under vacuum to afford the title compound as a white solid (31.4 g, 48.6 mmol, 39.5% yield).

¹H-NMR 400 MHz (D₂O): δ ppm: 7.84 (s, 4H), 4.53 (br d, J=10.8 Hz, 2H), 3.54-3.36 (m, 2H), 3.35-3.26 (m, 8H), 3.07 (br dd, J=7.6, 12.2 Hz, 2H), 2.53-2.51 (m, 2H), 2.19-1.98 (m, 4H), 1.94-1.91 (m, 6H).

LCMS (Method 1): Rt=2.275 min, MS m/z [M+H]⁺ 499.4, theoretical mass: 498.6

HPLC (Method 1): Rt=3.908 min, 99.7%

Elemental Analysis: C, 45.89%; H, 7.96%; N, 8.18%, theoretical+10 H₂O: C, 46.01%; H, 8.02%; N, 8.25%.

15 mg compound 4A was dissolved in 1.2 ml ethanol/H₂O (1:1) at 60° C. The solution was filtered through 0.45 m microporous filter and kept in a sealed 4 ml vial at room temperature. Needle crystals were observed in the solution and a crystal selected for X-ray crystallographic analysis

The crystal was a colorless needle with the following dimensions: 0.30×0.04×0.04 mm3. The symmetry of the crystal structure was assigned the orthorhombic space group C222₁ with the following parameters: a=15.9948(2) Å, b=22.5673(3) Å, c=9.5013(2) Å, α=90°, β=90°, γ=90°, V=3429.58(10) Å 3, Z=4, Dc=1.280 g/cm 3, F(000)=1424.0, μ(Cu Kα)=0.889 mm⁻¹, and T=110(14) K using Rigaku Oxford Diffraction XtaLAB Synergy four-circle diffractometer equipped with a HyPix-6000HE area detector. Cryogenic system: Oxford Cryostream 800 Cu: λ=1.54184 Å, 50 W, Micro focus source with multilayer mirror (μ-CMF). Distance from the crystal to the CCD detector: d=35 mm Tube Voltage: 50 kV Tube Current: 1 mA.

The absolute configuration of Example 1 was assigned (R,R, R,R)

### Step 3 Preparation of HCl Salt

To the suspension of the free parent (Step 2, 30.9 g, 45.5 mmol, 1 eq, 10H₂O) in H₂O (760 mL) and EtOH (760 mL) was added HCl (12 M, 7.61 mL, 2.01 eq) at 25° C. and stirred for 12 hr. The reaction mixture was concentrated under vacuum. The bis HCl salt (28.2 g, 39.0 mmol, 85.7% yield, 10H₂O) was obtained as a crystalline off-white solid.

LCMS (Method 1): Rt=2.300 min, MS m/z 250.1 [M+H/2]⁺

HPLC (Method 2): Rt=3.889 min, 99.3%

¹H-NMR 400 MHz (D₂O): δ ppm: 7.85 (s, 4H), 4.67 (d, J=11.2 Hz, 2H), 3.63-3.50 (m, 2H), 3.41-3.22 (m, 8H), 3.10 (ddd, J=1.8, 6.8, 13.2 Hz, 2H), 2.73-2.58 (m, 2H), 2.30-2.16 (m, 4H), 2.10-1.88 (m, 6H).

### EXAMPLES

In the following detailed description, each Example is a prodrug of Formula (I) that suitably in vivo release the respective Reference Example compounds of Formula (III).

### Example 1 (APL-7068)

### (R,2R,2′R)-2,2′-(terephthaloylbis(azanediyl))bis(2-((R)-quinuclidin-3-yl)acetic acid) bis methyl ester

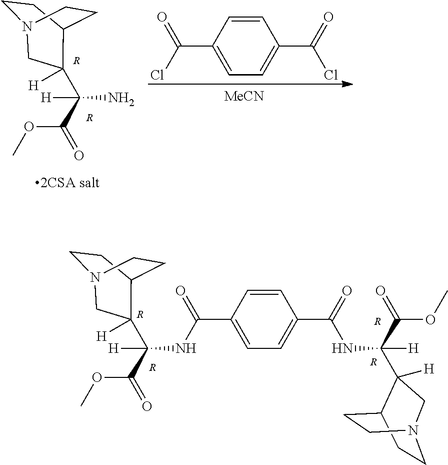

To a mixture of bis CSA salt compound 5 (5 g, 7.5 mmol) in MeCN (20 mL) was added DIPEA (2.54 g, 19.6 mmol) resulting in a brown solution. The reaction mixture was heated to 50° C. and a suspension of 1,4-terephthaloyl chloride (0.67 g, 3.3 mmol) in MeCN (20 mL) was added dropwise over 20 minutes. The reaction mixture was further stirred for 2 hours at 50° C. EtOAc (30 mL) was added followed by a 20% (aq) potassium carbonate solution (40 mL). Further water (10 mL) and MeCN (10 mL) were added and the aqueous layer was separated. Further 20% (aq) potassium carbonate solution (40 mL) and MeCN (10 mL) was added to the organic layer and the resulting aqueous layer collected. The precipitate present during the extraction was filtered to afford an off-white solid and collected as the title compound (280 mg).

¹H NMR (CD₃OD) δ ppm: 7.95 (s, 4H), 4.75-4.70 (m, 2H), 3.75 (s, 6H), 3.20-3.10 (m, 2H), 2.90-2.75 (m, 8H), 2.60-2.50 (m, 2H), 2.25-2.20 (m, 2H), 2.05-1.90 (m, 2H), 1.80-1.68 (m, 4H), 1.65-1.50 (m, 4H).

LCMS Rt=4.13 minutes, MS m/z 527 [M+H]⁺, 264 [M+H]/2⁺

### Reference Example 1

### (R,2R,2′R)-2,2′-(terephthaloylbis(azanediyl))bis(2-((R)-quinuclidin-3-yl)acetic acid)

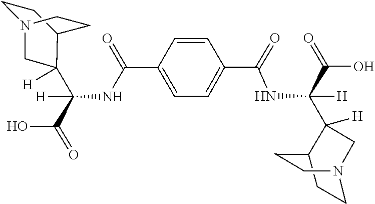 [SMILES: O=C(N[C@@H](C(=O)O)C1CN2CCC1CC2)c1ccc(C(=O)N[C@@H](C(=O)O)C2CN3CCC2CC3)cc1]

To a solution of the Example 1 bis methyl ester (180.00 mg, 341.80 μmol) in THF (2.00 mL) was added a solution of LiOH (48.00 mg, 2.00 mmol) in water (2 mL) at 25° C. The mixture was stirred for 1 hour at 25° C. The mixture was concentrated under reduced pressure to remove THF. To the mixture was added 1M HCl (aq) to pH=3. The mixture was concentrated under reduced pressure. The residue was dissolved in MeOH (5 mL) and purified by preparative HPLC (TFA) to give Example 1 (24.20 mg, 48.05 μmol, 14.06% yield, 99% purity) as a white solid.

¹H NMR 400 MHz (D₂O): δ ppm 7.77 (s, 4H), 4.62 (d, J=11.2 Hz, 2H), 3.50 (br t, J=10.9 Hz, 2H), 3.41-3.14 (m, 8H), 3.11-2.96 (m, 2H), 2.65-2.57 (m, 2H), 2.32-2.07 (m, 4H), 2.05-1.79 (m, 6H).

LCMS: Rt=5.91, MS m/z 501.1 [M+H]⁺, theoretical mass: 500.2

The following Examples and Reference Examples were prepared using the General method below using the appropriate dicarboxylic acid as described for each Example and Reference Example, and compound 5 (R,R). The Examples were purified as individually described.

### General Method for Examples 2-11

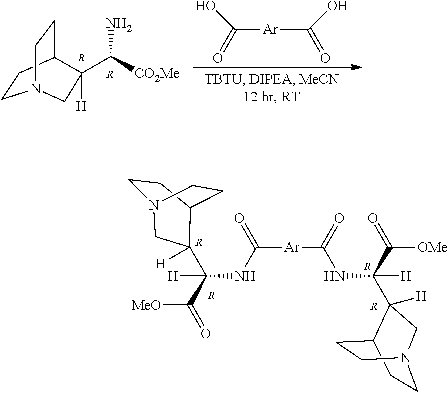

To a solution of compound 5 (2.70 eq) in ACN (10 V) was added TBTU (2.23 eq) and the appropriate carboxylic acid (1 eq) at 20° C. under nitrogen. DIEA (4.11 eq) was added to the mixture, and the mixture was stirred at 20° C. for 6 hrs under N₂. The reaction mixture was concentrated under vacuum and purified as described for each Example.

### General Method for Reference Examples 2-11

To a solution of the bis-methyl esters (1.00 eq) in IPA (20.0 V) was added aqueous KOH (10.0%, 10.0 eq) at 20° C. The mixture was stirred at 50° C. for 1 hr, cooled to room temperature and purified as described for each Reference Example.

### Example 2 APL-6968 Bis Methyl Ester

### (R,2R,2′R)-2,2′-((pyridine-2,5-dicarbonyl)bis(azanediyl))bis(2-((R)-quinuclidin-3-yl)acetic acid) bis methyl ester

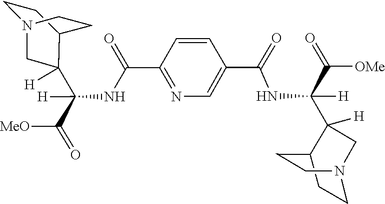 [SMILES: COC(=O)[C@H](NC(=O)c1ccc(C(=O)N[C@@H](C(=O)OC)C2CN3CCC2CC3)nc1)C1CN2CCC1CC2]

Example 2 was prepared according to the General Method using pyridine-2,5-dicarboxylic acid.

The residue was purified by prep-HPLC (column: Phenomenex Gemini-NX C18 75×30 mm, 3 μm; mobile phase: [water (0.225% FA)-ACN]; B %: 1%-20%, 7 min) to give the bis methyl ester (330 mg, 524 μmol, 43.8% yield, 91.2% purity, FA) as a white solid.

¹H-NMR 400 MHz (CDCl₃): δ ppm: 8.93 (br s, 1H), 8.82-8.60 (m, 2H), 8.50-8.40 (m, 2H), 8.26-8.24 (m, 1H), 8.02-8.01 (m, 1H), 4.92-4.84 (m, 1H), 4.82-4.73 (m, 1H), 3.80 (d, J=14.0 Hz, 6H), 3.28-3.12 (m, 7H), 2.31-2.12 (m, 12H), 1.99-1.77 (m, 10H).

LCMS (Method 1) Rt=0.685 min, MS m/z [M+H]⁺ 528.2

### Reference Example 2 APL-6968

### (R,2R,2′R)-2,2′-((pyridine-2,5-dicarbonyl)bis(azanediyl))bis(2-((R)-quinuclidin-3-yl)acetic acid)

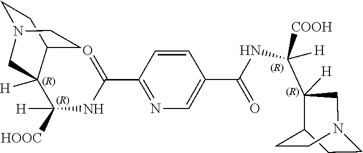

The residue was purified by prep-HPLC (column: Waters Atlantis T3 150×30 mm, 5 μm; mobile phase: [water (0.225% FA)-ACN]; B %: 1%-20%, 10 min) to afford Reference Example 2 (74.0 mg, 132 μmol, 97.4% purity, FA) as a white solid.

MS (Method 8): MS m/z 499.9 [M+H]⁺, theoretical mass: 499.2

HPLC (Method 1): Rt=2.31 min

¹H-NMR 400 MHz (D₂O): δ ppm: 8.97 (d, J=1.6 Hz, 1H), 8.43 (s, 1H), 8.33-8.31 (m, 1H), 8.13 (d, J=8.0 Hz, 1H), 4.56 (dd, J=8.4, 10.4 Hz, 2H), 3.60-3.52 (m, 2H), 3.39-3.24 (m, 8H), 3.13-3.04 (m, 2H), 2.62-2.51 (m, 2H), 2.28-2.19 (m, 4H), 2.05-1.89 (m, 6H).

### Example 3 APL-6969 Bis Methyl Ester

### (R,2R,2′R)-2,2′-((pyrazine-2,5-dicarbonyl)bis(azanediyl))bis(2-((R)-quinuclidin-3-yl)acetic acid) bis methyl ester

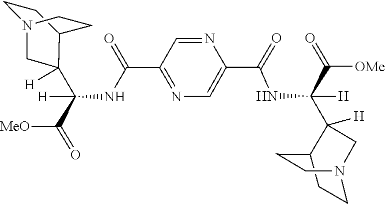 [SMILES: COC(=O)[C@H](NC(=O)c1cnc(C(=O)N[C@@H](C(=O)OC)C2CN3CCC2CC3)cn1)C1CN2CCC1CC2]

Example 3 was prepared according to the General Method using pyrazine-2,5-dicarboxylic acid.

The residue was purified by prep-HPLC (column: Waters Xbridge 150×25 mm, 5 μm; mobile phase: [water (10 mM NH₄HCO₃)-ACN]; B %: 14%-44%, 9 min) to give the bis methyl ester (90.0 mg, 124 μmol, 10.4% yield, 72.7% purity) as a white solid.

LCMS (Method 1): Rt=0.704 min, MS m/z 529.3 [M+H]⁺

### Reference Example 3 APL-6969

### (R,2R,2′R)-2,2′-((pyrazine-2,5-dicarbonyl)bis(azanediyl))bis(2-((R)-quinuclidin-3-yl)acetic acid)

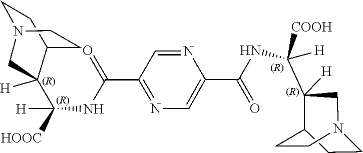

The mixture was filtered, FA (aq, 20% in water) was added to adjust the mixture to pH=7-8 and the mixture was purified by prep-HPLC (column: Waters Xbridge 150×25 mm, 5 μm; mobile phase: [water (10 mM NH₄HCO₃)-ACN]; B %: 1%-10%, 9 min) to give Reference Example 3 (51.0 mg, 98.0 μmol, 57.5% yield, 96.0% purity) as a white solid.

MS (Method 2): MS [M+H]⁺ 501.1, theoretical mass: 500.2

HPLC (Method 3): Rt=0.824 min

¹H-NMR 400 MHz (D₂O): δ ppm: 9.21 (s, 2H), 8.38 (br s, 4H), 4.55 (d, J=3.60 Hz, 2H), 3.53-3.48 (m, 2H), 3.40-3.20 (m, 8H), 3.10-3.01 (m, 2H), 2.63-2.52 (m, 2H), 2.21 (br s, 4H), 2.07-1.85 (m, 6H).

### Example 4 APL-6970 Bis Methyl Ester

### (R,2R,2′R)-2,2′-((pyridazine-3,6-dicarbonyl)bis(azanediyl))bis(2-((R)-quinuclidin-3-yl)acetic acid) bis methyl ester

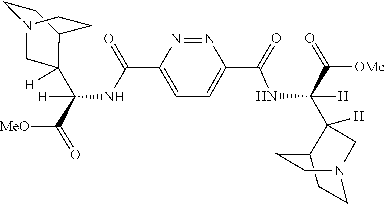 [SMILES: COC(=O)[C@H](NC(=O)c1ccc(C(=O)N[C@@H](C(=O)OC)C2CN3CCC2CC3)nn1)C1CN2CCC1CC2]

Example 4 was prepared according to the General Method using pyridazine-3,6-dicarboxylic acid.

The residue was purified by prep-HPLC (column: Phenomenex Gemini-NX C18 75×30 mm 3 μm; mobile phase: [water (10 mM NH₄HCO₃)-ACN]; B %: 5%-35%, 8 min) to give the bis methyl ester (110 mg, 144 μmol, 22.0% yield, 81.1% purity) as a white solid.

¹H-NMR 400 MHz (CDCl₃): δ ppm: 7.86 (d, J=8.4 Hz, 2H), 7.72 (s, 1H), 7.65 (dd, J=1.6, 8.0 Hz, 1H), 7.40 (d, J=8.4 Hz, 2H), 7.29 (d, J=8.0 Hz, 1H), 6.55-6.50 (m, 2H), 4.96-4.91 (m, 2H), 3.79 (s, 6H), 3.16-3.05 (m, 3H), 3.02-2.69 (m, 11H), 2.31 (s, 3H), 2.09-1.87 (m, 9H), 1.79-1.63 (m, 5H).

LCMS (Method 1): Rt=0.807 min, MS m/z 617.3

### Reference Example 4 APL-6970

### (R,2R,2′R)-2,2′-((pyridazine-3,6-dicarbonyl)bis(azanediyl))bis(2-((R)-quinuclidin-3-yl)acetic acid)

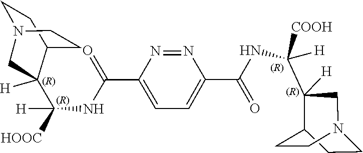

The residue was purified by prep-HPLC (column: Waters Atlantis T3 150×30 mm, 5 μm; mobile phase: [water (0.225% FA)-ACN]; B %: 1%-20%, 10 min) to give Reference Example 4 (87.0 mg, 154 μmol, 32.7% yield, 97.3% purity, FA) as a white solid.

LCMS (Method 8): Rt=2.296 min, MS m/z 501.4 [M+H]⁺, theoretical mass: 500.3

¹H-NMR 400 MHz (D₂O): δ ppm: 8.41 (s, 2H), 8.35 (s, 0.25H), 4.62 (d, J=10.8 Hz, 2H), 3.62-3.52 (m, 2H), 3.42-3.22 (m, 8H), 3.19-3.09 (m, 2H), 2.68-2.55 (m, 2H), 2.30-2.18 (m, 4H), 2.09-1.86 (m, 6H).

### Example 5 APL-6971 Bis Methyl Ester

### (R)-2-(4′-(((R)-carboxy((R)-quinuclidin-3-yl)methyl)carbamoyl)-[1,1′-biphenyl]-4-ylcarboxamido)-2-((R)-quinuclidin-3-yl)acetic acid bis methyl ester

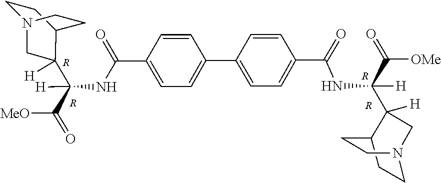

Example 5 was prepared according to the General Method using [1,1′-biphenyl]-4,4′-dicarboxylic acid.

The crude material was obtained as a colorless liquid.

LCMS (Method 1) Rt=0.789 min, MS m/z 603.4 [M+H]⁺

### Reference Example 5 APL-6971

### (R)-2-(4′-(((R)-carboxy((R)-quinuclidin-3-yl)methyl)carbamoyl)-[1,1′-biphenyl]-4-ylcarboxamido)-2-((R)-quinuclidin-3-yl)acetic acid

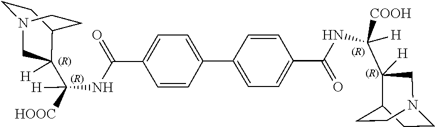

The mixture was filtered, and the FA (aq, 20% in water) was added the mixture adjust to pH=7-8. Purified by prep-HPLC (column: Waters Xbridge 15×25 mm, 5 μm; mobile phase: [water (10 mM NH₄HCO₃)-ACN]; B %: 1%-10%, 9 min) to give Reference Example 5 (37.0 mg, 63.7 μmol, 15.5% yield, 99.0% purity) as a white solid.

LCMS (Method 4): Rt=1.43 min, MS m/z 575.3 [M+H]⁺, theoretical mass: 574.2

¹H-NMR 400 MHz (D₂O): δ ppm: 7.89-7.77 (m, 8H), 4.55 (d, J=10.8 Hz, 2H), 3.59-3.50 (m, 2H), 3.42-3.18 (m, 8H), 3.13-3.02 (m, 2H), 2.58-2.48 (m, 2H), 2.30-2.15 (m, 4H), 2.09-1.84 (m, 6H).

### Example 6 APL-6972 Bis Methyl Ester

(R)-2-(4′-(((R)-carboxy((R)-quinuclidin-3-yl)methyl)carbamoyl)-2′-methyl-[1,1′-biphenyl]-4-ylcarboxamido)-2-((R)-quinuclidin-3-yl)acetic acid bis methyl ester

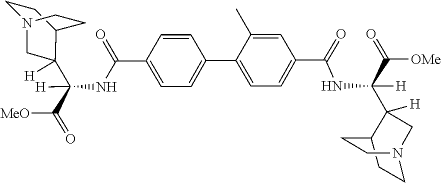 [SMILES: COC(=O)[C@H](NC(=O)c1ccc(-c2ccc(C(=O)N[C@@H](C(=O)OC)C3CN4CCC3CC4)cc2C)cc1)C1CN2CCC1CC2]

Example 6 was prepared according to the General Method using 2-methyl-[1,1′-biphenyl]-4,4′-dicarboxylic acid.

The residue was purified by prep-HPLC (column: Phenomenex Gemini-NX C18 75×30 mm, 3 μm; mobile phase: [water (10 mM NH₄HCO₃)-ACN]; B %: 5%-35%, 8 min) to give the bis ester (110 mg, 144 μmol, 22.0% yield, 81.1% purity) as a white solid.

LC-MS (Method 1): Rt=0.807 min, MS m/z 617.3 [M+H]⁺

¹H-NMR 400 MHz (CDCl₃): δ ppm: 7.86 (d, J=8.4 Hz, 2H), 7.72 (s, 1H), 7.65 (dd, J=1.6, 8.0 Hz, 1H), 7.40 (d, J=8.4 Hz, 2H), 7.29 (d, J=8.0 Hz, 1H), 6.55-6.50 (m, 2H), 4.96-4.91 (m, 2H), 3.79 (s, 6H), 3.16-3.05 (m, 3H), 3.02-2.69 (m, 11H), 2.31 (s, 3H), 2.09-1.87 (m, 9H), 1.79-1.63 (m, 5H).

### Reference Example 6 APL-6972

### (R)-2-(4′-(((R)-carboxy((R)-quinuclidin-3-yl)methyl)carbamoyl)-2′-methyl-[1,1′-biphenyl]-4-ylcarboxamido)-2-((R)-quinuclidin-3-yl)acetic acid

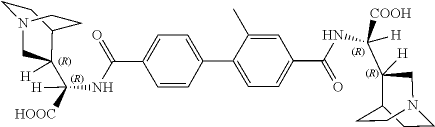

The residue was purified by prep-HPLC (column: Waters Atlantis T3 150×30 mm, 5 μm; mobile phase: [water (0.225% FA)-ACN]; B %: 1%-20%, 10 min) to give Reference Example 6 (FA salt, 16 mg, 5.44 μmol, 4.13% yield, 95.0% purity) as a white solid.

LCMS (Method 4): Rt=1.597 min, MS m/z 589.3 [M+H]⁺, theoretical mass: 588.3

¹H-NMR 400 MHz (D₂O+DMSO): δ ppm: 8.24 (s, 1H), 7.90-7.80 (m, 2H), 7.71 (s, 1H), 7.68-7.62 (m, 1H), 7.43 (br d, J=8.4 Hz, 2H), 7.34-7.27 (m, 1H), 3.35-3.32 (m, 2H), 3.26-3.05 (m, 9H), 2.91-2.83 (m, 2H), 2.22 (s, 3H), 2.15-2.07 (m, 4H), 1.92-1.68 (m, 7H).

### Example 7 APL-6973 Bis Methyl Ester

### (R,2R,2′R)-2,2′-((naphthalene-2,6-dicarbonyl)bis(azanediyl))bis(2-((R)-quinuclidin-3-yl)acetic acid) bis methyl ester

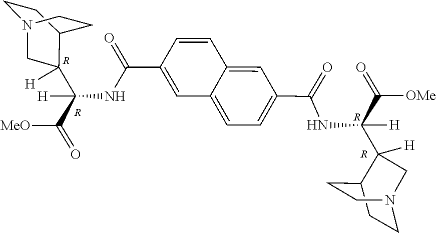

Example 7 was prepared according to General Method 1 using naphthalene-2,6-dicarboxylic acid.

The residue was purified by prep-HPLC (column: 3_Phenomenex Luna C18 75×30 mm, 3 μm; mobile phase: [water (0.1% TFA)-ACN]; B %: 5%-35%, 7 min), and the mixture was lyophilized to give the bis ester (160 mg, 277 μmol, 60.0% yield) as a white solid.

LC-MS (Method 1): Rt=0.770 min, MS m/z [M+H]⁺ 577.4

### Reference Example 7 APL-6973

### (R,2R,2′R)-2,2′-((naphthalene-2,6-dicarbonyl)bis(azanediyl))bis(2-((R)-quinuclidin-3-yl)acetic acid)

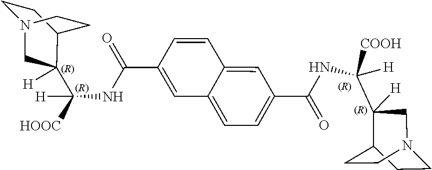

The mixture was filtered, and the FA (aq, 20% in water) was added the mixture adjust to pH 7-8. The residue was purified by prep-HPLC (column: Waters Xbridge 150*25 mm*5 um; mobile phase: [water (10 mM NH₄HCO₃)-ACN]; B %: 1%-10%, 9 min) to give Reference Example 7 (35.0 mg, 62.0 μmol, 22.0% yield, 96.0% purity) as a white solid.

LCMS (Method 5): Rt=0.282 min, MS m/z 549.1 [M+H]⁺, theoretical mass: 548.2

HPLC (Method 6): Rt=1.624 min

¹H-NMR 400 MHz (D₂O): δ ppm: 8.23 (s, 2H), 7.95 (d, J=10.0 Hz, 2H), 7.77 (d, J=10.0 Hz, 2H), 4.59 (d, J=11.2 Hz, 2H), 3.64-3.52 (m, 2H), 3.44-3.23 (m, 10H), 3.15-3.04 (m, 2H), 2.63-2.52 (m, 2H), 2.31-2.18 (m, 5H), 2.09-1.86 (m, 7H).

### Example 8 APL-6974 Bis Methyl Ester

### (R,2R,2′R)-2,2′-((2,5-dimethylterephthaloyl)bis(azanediyl))bis(2-((R)-quinuclidin-3-yl)acetic acid) bis methyl ester

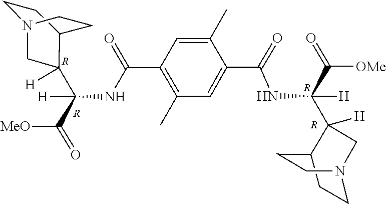

Example 8 was prepared according to the General Method using 2,5-dimethylbenzene-1,4-dicarboxylic acid.

The crude product was triturated with ACN (5 mL) and MeOH (3 mL) at 20° C. for 10 min and filtered to give the bis ester (114 mg, 185 μmol, 17.9% yield, 90.1% purity) as a white solid.

LCMS (Method 1): Rt=0.771 min, MS m/z 555.3 [M+H]⁺,

¹H-NMR 400 MHz (DMSO): δ ppm: 8.75 (d, J=6.8 Hz, 1H), 7.18 (br s, 1H), 4.50-4.46 (m, 1H), 3.68 (s, 3H), 3.17-2.91 (s, 12H), 2.78-2.68 (m, 1H), 2.30 (s, 3H), 2.20-2.19 (m, 1H), 1.96-1.90 (m, 1H), 1.83-1.68 (m, 2H), 1.74-1.53 (m, 2H), 1.16 (d, J=6.0 Hz, 1H).

### Reference Example 8 APL-6974

### (R,2R,2′R)-2,2′-((2,5-dimethylterephthaloyl)bis(azanediyl))bis(2-((R)-quinuclidin-3-yl)acetic acid)

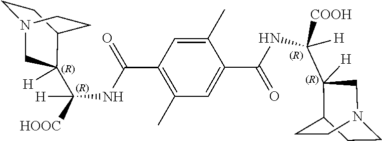

The residue was purified by prep-HPLC (column: Waters Atlantis T3 150×30 mm, 5 μm; mobile phase: [water (0.225% FA)-ACN]; B %: 1%-20%, 10 min) to give Reference example 8 (10.0 mg, 17.1 μmol, 9.50% yield, 98.1% purity, FA) as a white solid.

LCMS (Method 8): Rt=2.773 min, MS m/z 527.3 [M+H]⁺, theoretical mass: 526.3

¹H-NMR 400 MHz (D₂O+DMSO): δ ppm: 7.16 (s, 2H), 4.34 (br d, J=10.4 Hz, 2H), 3.41-3.33 (m, 2H), 3.22-3.10 (m, 7H), 2.94-2.88 (m, 2H), 2.36-2.27 (m, 3H), 2.21 (s, 6H), 2.16-2.01 (m, 4H), 1.90-1.70 (m, 6H).

LCMS m/z 527.3 [M+H]⁺, theoretical mass: 526.3, Rt=2.77 minutes, 100%

### Example 9 APL-6975 Bis Methyl Ester

### (R,2R,2′R)-2,2′-((2-methylterephthaloyl)bis(azanediyl))bis(2-((R)-quinuclidin-3-yl)acetic acid) bis methyl ester

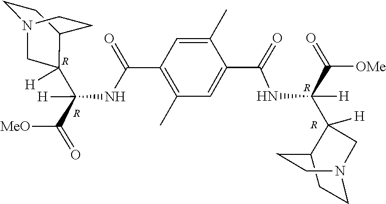

Example 9 was prepared according to the General Method using 2-methylbenzene-1,4-dicarboxylic acid.

The residue was purified by prep-HPLC (column: Phenomenex luna C18 150×25 mm, 10 μm; mobile phase: [water (0.225% FA)-ACN]; B %: 0%-20%, 10 min) to give the bis ester (500 mg, 647 μmol, 23.3% yield, 70.0% purity) as a white solid.

LCMS (Method 1): Rt=0.707 min, MS m/z 541.2 [M+H]⁺

### Reference Example 9 APL-6975

### (R,2R,2′R)-2,2′-((2-methylterephthaloyl)bis(azanediyl))bis(2-((R)-quinuclidin-3-yl)acetic acid)

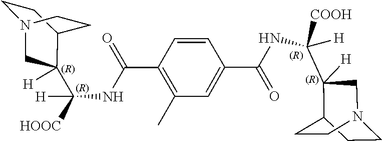

The mixture was filtered, and FA (aq, 20% in water) was added the mixture adjust to pH 7-8. The mixture was purified by prep-HPLC (column: Waters Xbridge 150×25 mm, 5 μm; mobile phase: [water (10 mM NH₄HCO₃)-ACN]; B %: 1%-10%, 9 min) to give Reference Example 9 (116 mg, 202.26 μmol, 54.67% yield, 97.4% purity, FA) as a white solid.

LCMS (Method 8): Rt=2.353 min, MS m/z 513.0 [M+H]⁺, theoretical mass: 512.3

¹H-NMR 400 MHz (D₂O): δ ppm: 8.38 (m, 1H), 7.64-7.59 (m, 2H), 7.42 (d, J=8.0 Hz, 1H), 4.53-4.48 (m, 2H), 3.60-3.49 (m, 2H), 3.39-3.21 (m, 8H), 3.12-2.99 (m, 2H), 2.54-2.40 (m, 2H), 2.35 (s, 3H), 2.30-2.14 (m, 4H), 2.04-1.86 (m, 6H).

### Example 10 APL-6976 Bis Methyl Ester

### (R,2R,2′R)-2,2′-((2,5-bis(benzyloxy)terephthaloyl)bis(azanediyl))bis(2-((R)-quinuclidin-3-yl)acetic acid) bis methyl ester

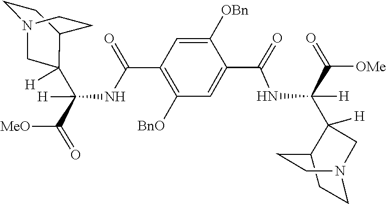 [SMILES: COC(=O)[C@H](NC(=O)c1cc(OCc2ccccc2)c(C(=O)N[C@@H](C(=O)OC)C2CN3CCC2CC3)cc1OCc1ccccc1)C1CN2CCC1CC2]

Example 10 was prepared according to the General Method using 2,5-bis(benzyloxy)benzene-1,4-dicarboxylic acid.

The residue was purified by prep-HPLC (column: Phenomenex luna C18 150×25 mm, 10 μm; mobile phase: [water (0.225% FA)-ACN]; B %: 11%-41%, 10 min to give the bis ester (180 mg, 211 μmol, 39.9% yield, 92.1% purity, FA) as a white solid.

¹H-NMR 400 MHz (CDCl₃): δ ppm: 8.52 (d, J=8.4 Hz, 2H), 8.37 (s, 1H), 7.93 (s, 2H), 7.58-7.40 (m, 10H), 5.27-5.17 (m, 4H), 4.79-4.75 (m, 2H), 3.69 (s, 6H), 3.34-3.14 (m, 6H), 3.07-2.95 (m, 2H), 2.90-2.75 (m, 4H), 2.11-2.01 (m, 2H), 1.99-1.88 (m, 6H), 1.85-1.70 (m, 4H).

### Reference Example 10 APL-6976

### (R,2R,2′R)-2,2′-((2,5-bis(benzyloxy)terephthaloyl)bis(azanediyl))bis(2-((R)-quinuclidin-3-yl)acetic acid)

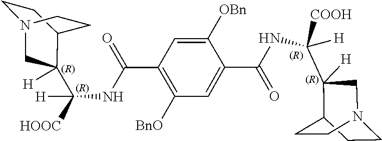

The residue was purified by prep-HPLC (column: Phenomenex luna C18 150×25 mm, 10 μm; mobile phase: [water (0.225% FA)-ACN]; B %: 1%-30%, 10 min) to give Reference Example 10 (67.0 mg, 87.6 μmol, 40.4% yield, 99.0% purity, FA) as a white solid.

LCMS (Method 1): Rt=0.799 min, MS m/z 711.3 [M+H]⁺, theoretical mass: 710.33

HPLC (method 7), Rt=2.519 min.

¹H-NMR 400 MHz (D₂O): δ ppm: 7.64 (s, 2H), 7.59-7.50 (m, 10H), 5.25-5.17 (m, 4H), 4.57 (d, J=10.4 Hz, 2H), 3.28-3.10 (m, 6H), 2.98-2.88 (m, 2H), 2.75-2.67 (m, 2H), 2.47-2.35 (m, 2H), 2.13-2.02 (m, 6H), 2.08-1.87 (m, 2H), 1.86-1.73 (m, 4H).

### Example 11 P2B-E Bis Methyl Ester

### 2-[[3-[[carboxy-[(3R)-quinuclidin-3-yl]methyl]carbamoyl]benzoyl]amino]-2-[(3R)-quinuclidin-3-yl]acetic acid bis methyl ester

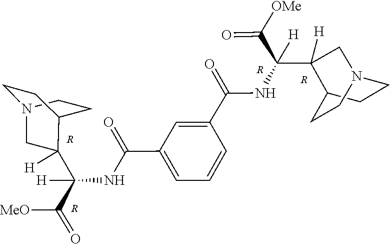

To a solution of methyl 2-amino-2-[(3R)-quinuclidin-3-yl]acetate (100.00 mg, 504.39 μmol) in CHCl₃ (4 mL) was added benzene-1,3-dicarbonyl chloride (51.20 mg, 252.20 μmol) at 30° C. The mixture was stirred for 16 hours at 30° C. The mixture was concentrated under reduced pressure to give a crude product as a yellow solid.

LCMS: Rt=0.881 min, MS m/z 527.3 [M+H]⁺

### Reference Example 11 P2B-E

### 2-[[3-[[carboxy-[(3R)-quinuclidin-3-yl]methyl]carbamoyl]benzoyl]amino]-2-[(3R)-quinuclidin-3-yl]acetic acid

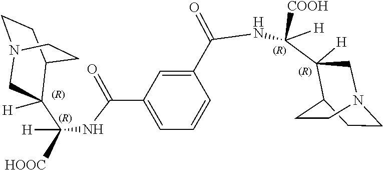

To a solution of the bis methyl ester (165.00 mg, 313.31 μmol) in THF (4 mL) was added LiOH (96.00 mg, 4.01 mmol, 12.79) in H₂O (4 mL) at 30° C. The mixture was stirred for 2 hours at 30° C. The mixture was concentrated under reduced pressure to remove THF. To the residue was added water (10 mL) and 1M HCl (aq) to pH=2. The mixture was concentrated under reduced pressure to give a crude product. The crude product was purified by preparative HPLC to afford Reference Example 11 (37.20 mg, 63.79 μmol, 40.72% yield, 98% purity, 2HCl) as a white solid.

¹H-NMR 400 MHz (D₂O): δ ppm: 8.10-8.01 (m, 1H), 7.88 (dd, J=1.7, 7.8 Hz, 2H), 7.54 (t, J=7.8 Hz, 1H), 4.62 (d, J=11.0 Hz, 2H), 3.59-3.50 (m, 2H), 3.38-3.15 (m, 8H), 3.11-2.99 (m, 2H), 2.71-2.54 (m, 2H), 2.26-2.07 (m, 4H), 2.06-1.78 (m, 6H).

LCMS Rt=5.9 min, MS m/z=499.3 [M+H]⁺, theoretical mass: 498.

### Example 12 (APL-7137)

### (R,2R,2′R)-2,2′-(terephthaloylbis(azanediyl))bis(2-((R)-quinuclidin-3-yl)acetic acid) bis ethyl ester

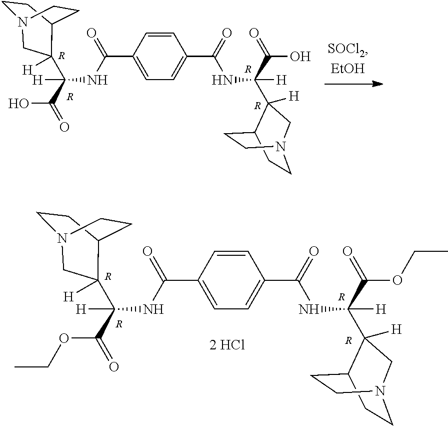

To a solution of the compound of Reference Example 1 (100 mg, 201 mol, 1.00 eq) in EtOH (10.0 mL) at 0° C. was added SOCl₂ (47.7 mg, 401 μmol, 29.1 uL, 2.00 eq). The suspension was stirred at 0-25° C. for 12 hrs. To the mixture was added saturated NaHCO₃ until pH=6-7. The mixture was filtered and the filtrate was concentrated. The residue was purified by prep-HPLC (column: Phenomenex luna C18 250*50 mm*10 um; mobile phase: [water (FA)-ACN]; B %: 1%-30%, 20 min) and lyophilized to give the title compound (51.4 mg, 79.2 umol, 39.5% yield, 99.6% purity, 2 FA) as a white solid.

¹H NMR (D₂O) δ ppm: 7.84 (s, 4H), 4.71 (br d, J=11.4 Hz, 2H), 4.24 (q, J=7.1 Hz, 4H), 3.45 (br t, J=11.7 Hz, 2H), 3.27-3.12 (m, 8H), 3.05-2.92 (m, 2H), 2.67-2.53 (m, 2H), 2.21-2.06 (m, 4H), 2.02-1.92 (m, 2H), 1.91-1.81 (m, 4H), 1.25 (t, J=7.1 Hz, 6H).

LCMS Rt=0.279 minutes, MS m/z 555.3 [M+H]⁺

HPLC Rt=0.88 minutes, 99.6% purity (220 nm)

### Example 13 (APL-7138)

### (R,2R,2′R)-2,2′-(terephthaloylbis(azanediyl))bis(2-((R)-quinuclidin-3-yl)acetic acid) bis isopropyl ester

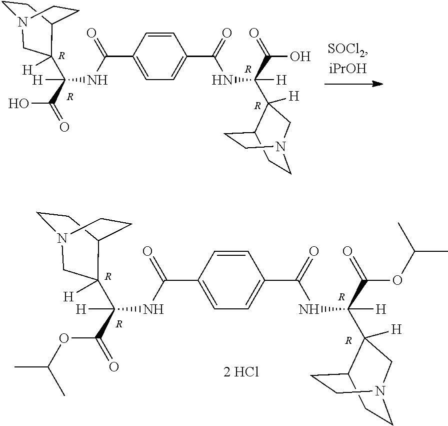

To a solution of the compound of Reference Example 1 (100 mg, 201 mol, 1.00 eq) in i-PrOH (10.0 mL) at 0-10° C. was added SOCl₂ (239 mg, 2.01 mmol, 145 L, 10.0 eq). The suspension was stirred at 75° C. for 12 hrs. The reaction was concentrated and solvent-swapped with MTBE (20.0 mL) twice to remove i-PrOH. The solid was dissolved with deionized water (20.0 mL) and lyophilized to give the title compound (84.6 mg, 123 umol, 61.5% yield, 95.6% purity, 2 HCl) as a yellow solid.

¹H NMR (D₂O) δ ppm: 7.84 (s, 4H), 5.07 (td, J=6.3, 12.5 Hz, 2H), 4.68 (d, J=11.3 Hz, 2H), 3.57 (br t, J=11.7 Hz, 2H), 3.41-3.25 (m, 8H), 3.12 (ddd, J=1.8, 6.7, 13.1 Hz, 2H), 2.76-2.63 (m, 2H), 2.29-2.15 (m, 4H), 2.09-1.92 (m, 6H), 1.28-1.21 (m, 12H)

LCMS Rt=0.31 minutes, MS m/z 583.3 [M+H]⁺

HPLC Rt=1.072 minutes, 95.6% purity (220 nm)

### Biological Assays

The following assays were performed on the Reference Examples (free drug forms of the prodrug) described above.

### MIRA Immunoturbidimetric Assay

The CRP immunoturbidimetric assay on the Roche COBAS MIRA Plus autoanalyser, utilises two different sized latex particles that are covalently coupled with two different monoclonal antibodies with specificity for different CRP epitopes (5). The assay was validated by Roche for measurement of native pentameric CRP, for which it has high sensitivity and specificity and a high upper detection limit. The method was calibrated against a standard produced in our laboratory. Serendipitously, one of the antibodies used in the assay binds to an epitope present on the ligand binding B face of CRP. Thus, when pairs of CRP molecules are cross linked by bivalent ligands to form B face to B face complexes in which the B faces are occluded, the assay fails to detect CRP. Other assays, using antibodies which bind to different epitopes, are unaffected by such cross linking. We deliberately designed the bivalent compounds such as BPC8 and APL-2191 to crosslink pairs of CRP pentamers in order to maximise blockade of CRP binding to the autologous ligands that triggers the pathogenicity of CRP. Therefore, inhibition of CRP recognition in the MIRA assay is a convenient tool to monitor the efficacy and potency of complex formation by such ligands (6).

CRP concentrations were measured in the presence and absence of ligands by the COBAS MIRA autoanalyser. Concentrated Tris-calcium buffer (×10 TC) was prepared in MilliQ water from trishydroxymethyamine (100 mM), calcium chloride (20 mM) and sodium chloride (1.4 M). The pH was adjusted to 8.0 using HCl and sodium azide was added (0.1% w/v); the buffer was stored at 4° C. A tenfold diluted working buffer (TC) was prepared by diluting 100 ml of the x10 concentrated buffer with 900 ml of MilliQ water. Human CRP was isolated, purified and characterised as previously reported (6-9) and stored frozen at −80° C. When required, stock CRP was rapidly thawed at 37° C. and used to prepare working dilutions that were kept at 4° C. for the duration of an experiment. CRP concentration was determined spectrophotometrically (Beckman Coulter DU 650) in quartz cuvettes with a 1 cm light path, by measuring A₂₈₀ with correction for light scattering by subtracting the A₃₂₀ absorbance at 320 nm ( ) and using the measured absorption coefficient A(1%, 1 cm)=17.5 for human CRP (10). Human CRP at ˜90 μg/ml (0.78 μM of pentamer) in TC buffer was prepared from a stock solution; a 75 μl aliquot was used in the assay. Compounds were supplied by Wuxi AppTec (Wuhan, China) as solids. They were dissolved in TC buffer at suitable concentrations, depending on solubility, of up to 10 mM (labelled S1). They were then serially diluted 1:2 with TC buffer (100 μl ligand+200 μl TC) to provide up to 9 dilutions, S2-S10. A TC buffer control (S0) was included in each assay. A 15 μl volume of each ligand solution was incubated with 75 μl of CRP for 1 h at room temperature. The final concentrations were 0.73 μM native pentameric CRP, ligands S1-S10=625-0.03 μM, corresponding to molar ligand:CRP ratios of 850-0.04. Where compounds were of reduced solubility in TC buffer, lower stock concentrations were used (from 0.6 mM), corresponding to final top assay concentrations of 100 μM.

Data are expressed as measured CRP (mg/L) against final total ligand concentration (μM) and were plotted in Sigmaplot (V14) using the 4 parameter logistic curve y=min+(max-min)/(1+(x/EC₅₀)⁻Hill slope) to calculate EC₅₀. Where appropriate, samples were also measured in whole normal human serum following the addition of a known amount of human CRP. All compounds were assayed in comparison with a highly purified preparation of bis(phosphocholine)octane (BPC8), that was prepared by Carbogen AMCIS AG and diluted into sterile water at 10 mM concentration. It was stored at −80° C. The solution was diluted into TC buffer as required.

Table 1 shows the data for the MIRA immunoturbidimetric assay for Reference Examples 1-12

TABLE 1

| Reference | | |
|---|---|---|
| Example No. | Compound ID | IC50 (μM) |
| 1 | APL-2191 | 0.60 |
| 2 | APL-6968 | 0.65 |
| 3 | APL-6969 | 1.22 |
| 4 | APL-6970 | 0.58 |
| 5 | APL-6971 | 0.73 |
| 6 | APL-6972 | 0.56 |
| 7 | APL-6973 | 0.51 |
| 8 | APL-6974 | 0.59 |
| 9 | APL-6975 | 0.55 |
| 10 | APL-6976 | 0.67 |
| 11 | P2B-E | 2.02 |
| 12 | BPC8 | 1.2 |

The Examples of Formula (I) and Reference Examples of Formula (III) are RR,RR stereoisomers. The other stereoisomers of this structure have lesser or no activity. The SS,SS isomer is the most active alternative isomer (denoted QA,QA Quinuclidine, Amino Acid: SS,SS IC50 34.4 μM, RS,RS IC50>1000 μM, SR,SR IC50>1000 μM, RS,RR>1000 μM). Alternative isomers may be prepared by one skilled in the art according to the methods above using the desired stereoisomers with a suitable protecting group strategy employed.

### Physicochemical Properties

### (A) Solubility

### Protocol for Determining Solubility

A known quantity of the Examples and control compounds were weighed into a suitable vessel (whatman miniuniprep vials) and a known volume of the required media was added (e.g. Simulated Gastric Fluid pH 1.2 [SGF], Simulated Fasted State Intestinal Fluid pH 6.5 [FaSSIF] or Phosphate Buffered Saline pH 7.4 [PBS]). The samples were subjected to vortex mixing for 2 minutes followed by agitation on a shaker for 24 hours at room temperature at 800 rpm. The samples were then centrifuged for 20 minutes at 4000 rpm, the miniuniprep vials compressed and the filtrates analysed by HPLC (Waters Xbridge C18 4.6×100 mm, mobile phase A: 0.1% TFA in water, mobile phase B: 0.1% TFA in acetonitrile). The parent compound APL-2191 is included as a comparison; APL-2191 is present as a bis HCl salt, which can significantly alters the pH profile as shown by the pH measurements taken before and after addition of the samples to the media. However, the results indicate that, overall, the examples are more soluble than the parent compound.

The solubility of the Examples in various aqueous media are shown below in Table 2:

TABLE 2
Solubility of Examples in PBS, FaSSIF and SGF at 24 hour timepoint.

| | | | | Thermo- |
|---|---|---|---|---|
| | | | | dynamic |
| | | Initial/final | | solubility |
| Ex No | R1/R2 | pH | Media | (μg/mL) |
| Ex 1 APL-7068 | OMe | 7.36/8.01 | PBS | 1219 |
| | | 6.46/7.22 | FaSSIF | 4686 |
| | | 1.23/1.33 | SGF | 5928 |
| Ex 2 APL-7137 | OEt | 7.36/7.49 | PBS | 5516 |
| | | 6.49/6.99 | FaSSIF | 4538 |
| | | 1.22/1.29 | SGF | 4004 |
| Ex 3 APL-7138 | OⁱPr | 7.36/7.35 | PBS | 4561 |
| | | 6.49/6.22 | FaSSIF | 5204 |
| | | 1.22/1.15 | SGF | 5142 |
| Parent APL- | OH | 7.36/6.44 | PBS | 1693 |
| 2191.2HCl | | 6.46/2.85 | FaSSIF | 2510 |
| | | 1.23/1.17 | SGF | 4557 |

Therefore the Examples are soluble across the pH range in biologically relevant media.

### (B) Stability

### Stock Solution Preparation:

### Examples

10 μL of a 10 mM stock solution of the Examples were dissolved in 490 μL DMSO to afford 200 μL working solution

### SGF (pH 1.2):

Dissolve 0.04 g NaCl and 0.064 g pepsin in 0.14 mL HCl and add sufficient water to 20 mL volume. The pH of the test solution was 1.20±0.05.

### FaSSIF (pH 6.5):

0.056% (w/v) lecithin, 0.161% (w/v) sodium taurocholate, 0.39% (w/v) monobasic potassium phosphate, 0.77% (w/v) potassium chloride, deionized H₂O. The pH of the test solution was 6.5±0.05.

### PBS (pH 7.4):

30 mL of 10×PBS (pH7.4) was added into a 50 mL flask and diluted with water. The solution was mixed well and adjusted to pH 7.4 with 1N HCl.

Protocol for Determining Stability 2 μL of the 200 μL working solution of the Examples was added into 96-deep-well plates for T0, T60, T120, T360 and T1440 timepoints. Duplicates were prepared.

Stability studies were commenced by the addition of 198 μL of the required media (e.g. Simulated Gastric Fluid pH 1.2 [SGF], Simulated Fasted State Intestinal Fluid pH 6.5 [FaSSIF]) and Phosphate Buffered Saline pH 7.4 [PBS]) to each well except T0. The samples were incubated at 37° C. at 600 rpm for the appointed time. The samples at the corresponding timepoint were removed and immediately mixed with 400 μL of 0.3% FA in MeCN containing 200 ng/mL tolbutamide and labetalol (internal standard). 200 μL of suspension was removed and mixed with 400 μL of 0.3% FA in cold MeCN containing 200 ng/mL tolbutamide and labetalol. The samples were centrifuged at 4000 rpm at 4° C. for 20 minutes. 60 μL of the supernatant was removed and mixed with 180 μL purified water and thoroughly mixed and analysed by LCMS (Shimadzu LC 30-AD, ACQUITY UPLC HSS T3 1.8 um 2.1*50 mm Part No. 186003538, with mobile phase A: 0.1% formic acid in water, mobile phase B: 0.1% formic acid in MeCN. Formation of parent was also monitored as a metabolite.

Samples were analysed by Mass Spectrometry QTRAP 6500+

The stability of the Examples in various aqueous media are shown below in Table 2:

TABLE 2
Stability of Examples in FaSSIF and SGF at 6 and 24 hour
timepoint. % remaining at chosen timepoint. For SGF and
FaSSIF, no parent was detected as a metabolite indicating
full stability. For PBS, parent was detected over the
period of time from 6 h to 24 h for APL-7068.

| | | FaSSIF | | SGF | | PBS | |
|---|---|---|---|---|---|---|---|
| | | (pH 6.5) | | (pH 1.2) | | (pH 7.4) | |
| Example | | % remaining | | % remaining | | % remaining | |
| Number | R1/R2 | 6 hr | 24 h | 6 h | 24 h | 6 h | 24 h |
| Example 1 | OMe | 98.3 | 113.3 | 101.2 | 92.6 | 84.07 | 55.89 |
| APL-7068 | | | | | | | |
| Example 12 | OEt | 105.0 | 95.2 | 101.2 | 92.3 | 84.33 | 87.14 |
| APL-7137 | | | | | | | |
| Example 13 | OiPr | 99.7 | 110.4 | 107.2 | 100.0 | 103.43 | 104.61 |
| APL-7138 | | | | | | | |

Each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

### REFERENCES

1. Pepys M. B. (2018) “The Pentraxins 1975-2018: Serendipity, Diagnostics and Drugs.” Front. Immunol. 9: 2382. doi: 10.3389/fimmu.2018.02382.
2. Griselli, M. Herbert, J., Hutchinson, W. L., Taylor, K. M., Sohail, M., Krausz, T. and Pepys, M. B., (1999) “C-reactive protein and complement are important mediators of tissue damage in acute myocardial infarction.” J. Exp. Med. 190: 1733-1739.
3. Gill, R., Kemp, J. A., Sabin, C. and Pepys, MB (2004) “Human C-Reactive Protein Increases Cerebral Infarct size after middle cerebral artery occlusion in adult rats.” J. Cereb. Blood Flow Metab. 24: 1214-1218.
4. Pepys, M. B., Hirschfield, G. M., Tennent, G. A., Gallimore, J. R., Kahan, M. C., Bellotti, V., Hawkins, P. N., Myers, R. M., Smith, M. D., Polara, A., Cobb, A. J. A., Ley, S. V., Aquilina, J. A., Robinson, C. V., Sharif, I., Gray, G. A., Sabin, C. A., Jenvey, M. C., Kolstoe, S. E., Thompson, D. and Wood, S. P. (2006) “Targeting C-reactive protein for the treatment of cardiovascular disease.” Nature 440: 1217-1221.
5. Eda, S., Kaufmann, J., Roos, W. and Pohl, S. (1998) “Development of a new microparticle-enhanced turbidometric assay for C-reactive protein with superior features in analytical sensitivity and dynamic range.” J. Clin. Lab. Anal. 12: 137-144
6. Pepys, M. B., Dash, A. C. and Ashley, J. (1977) “Isolation of C-reactive protein by affinity chromatography.” Clin. Exp. Immunol. 30: 32-37.
7. de Beer, F. C. and Pepys, M. B. (1982) “Isolation of human C-reactive protein and serum amyloid P component.” J. Immunol. Methods 50: 17-31.
8. Carlucci, F., Cook, H. T., Garg, A., Pepys, M. B. and Botto, M. (2010) “Lack of effect of a single injection of human C-reactive protein on murine lupus or nephrotoxic nephritis.” Arthritis Rheum. 62: 245-249.
9. Pepys, M. B., Gallimore, J. R., Lloyd, J., Li, Z., Graham, D., Taylor, G. W., Ellmerich, S., Mangione, P. P., Tennent, G. A., Hutchinson, W. L., Millar, D. J., Bennett, G., More, J., Evans, D., Mistry, Y., Poole, S. and Hawkins, P. N. (2012) “Isolation and characterization of pharmaceutical grade human pentraxins, serum amyloid P component and C-reactive protein, for clinical use.” J. Immunol. Methods 384: 92-102.
10. Nelson, S. R., Tennent, G. A., Sethi, D., Gower, P. E., Ballardie, F. W., Amatayakul-Chantler, S. and Pepys, M. B. (1991) “Serum amyloid P component in chronic renal failure and dialysis.” Clin. Chim. Acta 200(2-3): 191-200.

1. An agent for use in medicine, wherein the agent comprises a compound of Formula (I): 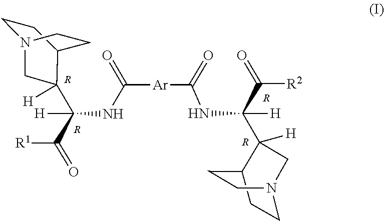
wherein: Ar is an aryl linker group, and
R¹ and R² are independently selected from groups of formula —OR³;
—O(CO)R³; —NR³R^{3′}; 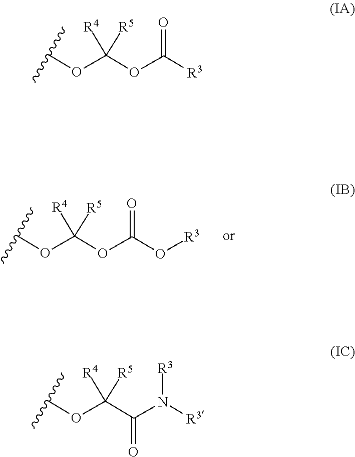
wherein:
R³ and R^{3′} are independently selected from H, C1-C12 alkyl, C1-C12 alkylene, C3-C8 (hetero)cycloalkyl, or C5-C12 (hetero)aryl, or R³ and R^{3′} together form a C3-C8 heterocyclic ring with the nitrogen, each optionally substituted with one, two or three groups selected from halogen, —CF₃, —OR⁶, —NR⁶R^{6′}, —COR⁶, and —CO₂R⁶, C3-C8 (hetero)cycloalkyl, or C5-C12 (hetero)aryl;
R⁴ and R⁵ are independently selected from H or C1-C6 alkyl; and
R⁶ and R^{6′} are independently C1-C6 alkyl, or together form a C3-C8 heterocyclic ring with the nitrogen,
provided that R¹ and R² are not both —OH,
including individual pharmaceutically acceptable salts, solvates, or derivatives thereof.

2. An agent for use in medicine according to claim 1, wherein the linker group Ar is selected from the group consisting of the following general Formulae Ar-I to Ar-VI: 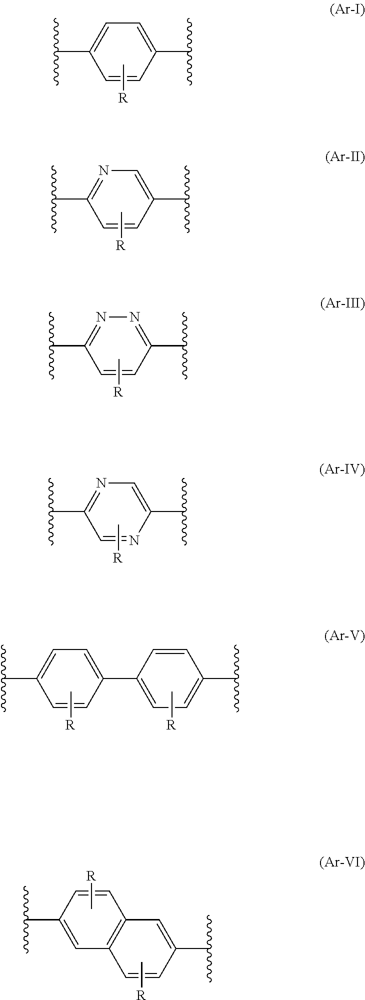
wherein R represents one or more optional substituents on the aryl ring(s) selected from halogen, hydroxy, cyano, —CONH₂, or C1-C5 (cyclo)alkyl or C1-C5 (cyclo)alkoxy wherein the alkyl groups are optionally substituted with a phenyl group or with one or more halogen atoms.

3. An agent for use in medicine according to claim 2, wherein the aryl linker group Ar is selected from the group consisting of groups having formulae Ar-VII to Ar-XVI:

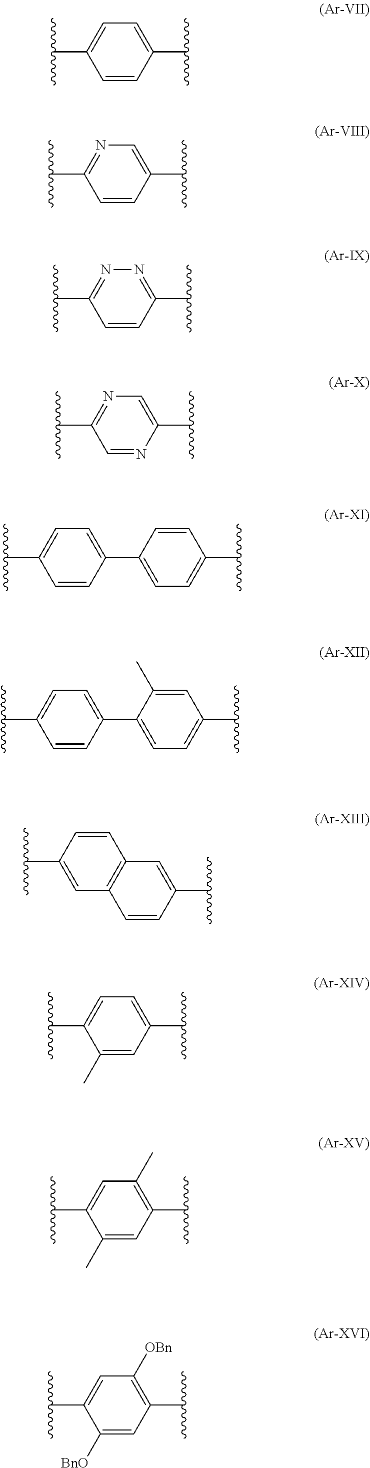

4. An agent for use in medicine according to any preceding claim, wherein the diastereomeric purity of the (R,R,R,R) isomer is at least about 50% by weight, suitably at least about 60%, more suitably at least about 75%, still more suitably at least about 90%, and most suitably at least about 98%.

5. An agent for use in medicine according to any preceding claim, wherein the compound of Formula (I) has the following Formula (II):

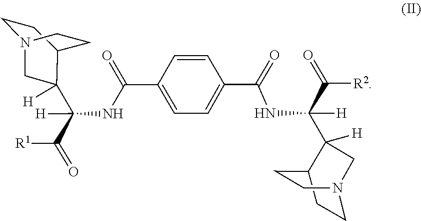

6. An agent for use in medicine according to any preceding claim, wherein neither R¹ nor R² is —OH; preferably wherein R¹ and R² are the same.

7. An agent for use in medicine according to any preceding claim, wherein R⁴ is H and R⁵ is H or C1-C3 alkyl

8. An agent for use in medicine according to any preceding claim, wherein the compound of Formula (I) is a hydrochloride salt, in particular a 0.2HCl salt.

9. An agent for use in medicine according to any preceding claim, wherein the compound of Formula (I) reacts in vivo in the mammalian body to release an agent of Formula (III):

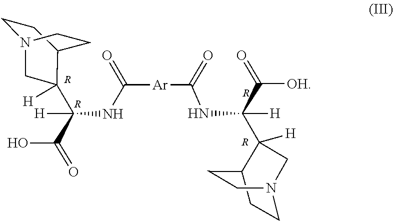

10. An agent for use in medicine according to claim 9, wherein the compound of Formula (III) is an inhibitor of human C-reactive protein (CRP) having an IC₅₀ of about 20μM or less, still more preferably about 10 μM or less, or about 5 μM or less, or about 1 μM or less.

11. An agent according to any preceding claim, for use in the treatment or prevention of tissue damage in a subject having an inflammatory and/or tissue damaging condition.

12. An agent according to claim 11, wherein the inflammatory and/or tissue damaging condition comprises one or more of acute coronary syndrome, unstable angina, plaque rupture, and/or incipient atherothrombosis.

13. An agent according to claim 12, wherein the inflammatory and/or tissue damaging condition is selected from an infection, an allergic complication of infection, an inflammatory disease, ischemic or other necrosis, traumatic tissue damage and malignant neoplasia.

14. An agent according to claim 13, wherein the condition is an infection selected from a bacterial infection including sepsis, a viral infection for example a severe acute respiratory syndrome (SARS) viral infection such as a SARS-Cov-2 infection, a fungal infection and a parasitic infection.

15. An agent according to claim 11, wherein the condition is an inflammatory disease selected from rheumatoid arthritis, juvenile chronic (rheumatoid) arthritis, ankylosing spondylitis, psoriatic arthritis, systemic vasculitis, polymyalgia rheumatica, Reiter's disease, Crohn's disease and familial Mediterranean fever and other autoinflammatory conditions.

16. An agent according to claim 11, wherein the condition is tissue necrosis selected from myocardial infarction, ischaemic stroke, tumour embolization and acute pancreatitis.

17. An agent according to claim 11, wherein the condition is trauma selected from elective surgery, burns, chemical injury, fractures and compression injury.

18. Use according to claim 11, wherein the condition is malignant neoplasia selected from lymphoma, Hodgkin's disease, carcinoma and sarcoma.

19. An agent according to claim 11, wherein the condition is an allergic complication of infection selected from rheumatic fever, glomerulonephritis, and erythema nodosum leprosum.

20. A pharmaceutical composition comprising an agent according to any of claims 1 to 10 in admixture with one or more pharmaceutically acceptable excipients, diluents or carriers.
